(19) United States
(12) Patent Application Publication
(10) Pub. No.: US 2026/0137753 A1
(43) Pub. Date: May 21, 2026

(54) DAPTOMYCIN COMPOSITIONS

(71) Applicant: MAIA Pharmaceuticals, Inc., Princeton, NJ (US)

(72) Inventors: Srikanth Sundaram, Somerset, NJ (US); Daniel Charles Stewart, Queensbury, NY (US)

(21) Appl. No.: 19/301,835

(22) Filed: Aug. 15, 2025

Related U.S. Application Data
(60) Provisional application No. 63/723,325, filed on Nov. 21, 2024.

(51) Int. Cl. A61K 38/12; A61K 9/00; A61K 9/08; A61K 9/16; A61K 9/19

(52) U.S. Cl. CPC A61K 38/12; A61K 9/0019; A61K 9/08; A61K 9/1611; A61K 9/1617; A61K 9/19

(57) ABSTRACT

The present disclosure provides daptomycin formulations which have improved chemical stability, faster reconstitution times when reconstituted from the solid state and longer in-use stability. The compositions comprise daptomycin, one basic amino acid or its salt and optionally one or more pH adjusting agents.

This application claims the benefit of U.S. Provisional Application No. 63/723,325, filed Nov. 21, 2024, which is hereby incorporated by reference.

### FIELD OF THE INVENTION

This disclosure relates to improved solid daptomycin compositions for reconstitution with a liquid diluent to form pharmaceutical compositions suitable for parenteral administration, as well as methods of making such solid compositions. This disclosure also relates to methods for the treatment of a bacterial infection in a subject in need thereof using the reconstituted daptomycin compositions.

### BACKGROUND OF THE INVENTION

Daptomycin is a cyclic lipopeptide antibiotic used to treat systemic and life-threatening infections caused by Gram-positive organisms. Daptomycin is available in the United States as a powder for injection, which is for intravenous administration for the treatment of infections caused by susceptible strains of multiple Gram-positive microorganisms. The bactericidal activity of daptomycin is attributed to its mechanism of action, which involves the rapid depolarization of the membrane potential in Gram-positive bacteria. This inhibits essential cellular processes, including DNA, RNA and protein synthesis, ultimately resulting in cell death.

Daptomycin marketed as CUBICIN® (daptomycin for injection) is approved in the U.S. for the treatment of (a) adult and pediatric patients (1 to 17 years of age) with complicated skin and skin structure infections (cSSSI) caused by susceptible isolates of the following Gram-positive bacteria: Staphylococcus aureus (including methicillin-resistant isolates), Streptococcus pyogenes, Streptococcus agalactiae, Streptococcus dysgalactiae subsp. equisimilis, and Enterococcusfaecalis (vancomycin-susceptible isolates only), (b) adult patients with Staphylococcus aureus bloodstream infections (bacteremia), including adult patients with right-sided infective endocarditis, caused by methicillin-susceptible and methicillin-resistant isolates, and (c) pediatric patients (1 to 17 years of age) with Staphylococcus aureus bloodstream infections (bacteremia).

The lyophilized powder of daptomycin in each CUBICIN® vial needs to be reconstituted with a diluent before parenteral administration. The reconstitution time for these lyophilized daptomycin compositions is more than 10 minutes in a pharmaceutical diluent, depending on the specific reconstitution method employed. According to U.S. Pat. No. 9,138,456, the reconstitution time can be 15-45 minutes depending on the reconstitution procedure.

U.S. Pat. Nos. 8,835,382 and 9,138,456 describe daptomycin formulations containing sucrose.

U.S. Pat. No. 9,655,946 discloses a lyophilized daptomycin formulation comprising an additive selected from the group consisting of pharmaceutically acceptable antioxidants, pharmaceutically acceptable organic acids and pharmaceutically acceptable salts thereof, pharmaceutically acceptable glucose derivatives and pharmaceutically acceptable salts thereof, and combinations thereof.

U.S. patent Ser. No. 11/759,497 describes a pharmaceutical composition containing daptomycin and at least one amino acid.

A significant shortcoming of previously commercially available daptomycin compositions is the long reconstitution time, typically taking from about 15 to about 45 minutes, depending on the specific reconstitution technique employed. See, e.g., U.S. Pat. No. 9,138,456. Such long reconstitution times are not ideal for therapeutic situations as regards ease and efficiency of administration. The long wait time for the lyophilizate to become reconstituted can also lead to issues with incomplete dissolution prior to administration, which can result in adverse effects for parenteral intravenous administration.

A number of daptomycin degradation-based impurities have been identified (see, e.g., U.S. Pat. No. 8,058,238). Major degradants of daptomycin include anhydrodaptomycin derivatives in which an α-aspartyl group is transpeptidated to an anhydrosuccinamido group, 3-isomer of daptomycin in which the compound contains a β-aspartyl group instead of an α-aspartyl group, and the lactone hydrolysis product of daptomycin in which one of the ester moieties is hydrolyzed.

There is a continuing need for solid daptomycin compositions that can be reconstituted rapidly (e.g., in less than about 5 minutes) in a pharmaceutically acceptable diluent to form reconstituted daptomycin formulations for parenteral administration. There is also a continuing need for solid lyophilized daptomycin compositions with improved chemical stability in the solid form, reconstituted form and/or diluted form. Such compositions may exhibit longer shelf life, less need for refrigerated storage, increased tolerance under various storage conditions, reduced handling time for reconstitution of the product before use and increased chemical stability after reconstitution as a liquid formulation for parenteral administration. Such formulations will provide for more rapid administration and more reliable dosing of daptomycin, due to the reduced impurity levels.

### SUMMARY OF THE INVENTION

An objective of the present invention is to provide a stable solid pharmaceutical composition comprising daptomycin.

Another objective of the present invention is to provide a solid pharmaceutical composition comprising daptomycin which is reconstituted using a suitable diluent in less than about 15 minutes.

Another objective of the present invention is to provide a stable reconstituted and diluted daptomycin composition.

Another objective of the present invention is to provide an industrially and economically viable process for the preparation of a stable daptomycin composition.

One embodiment of the present invention provides a pharmaceutical composition comprising of daptomycin, one basic amino acid or its salt (e.g., L-arginine or L-arginine HCl), and optionally one or more pH adjusting agents.

Another embodiment of the present invention provides a solid pharmaceutical composition (e.g., a solid lyophilized pharmaceutical composition) comprising of daptomycin, one basic amino acid or its salt, and one or more pH adjusting agents, wherein the molar ratio of daptomycin to the one basic amino acid is from about 1:3.5 to about 1:7, such as about 1:3.5, about 1:4, about 1:4.5, about 1:4.6, about 1:4.7, about 1:5, about 1:5.5, about 1:5.6, about 1:6, about 1:6.5 or about 1:7. In one embodiment, the molar ratio of daptomycin to the one or more basic amino acids or its salts (e.g., L-arginine or L-arginine HCl) is about 1:3.8 to about 1:5.6. In another embodiment, the molar ratio of daptomycin to the one or more basic amino acids or its salts (e.g., L-arginine or L-arginine HCl) is about 1:3.8 to about 1:5.5.

Another embodiment of the present invention provides a stable solid pharmaceutical composition (e.g., a solid lyophilized pharmaceutical compositions) comprising of daptomycin, one or more basic amino acids or its salts (e.g., L-arginine HCl), and one or more pH adjusting agents, wherein the pH of the composition is about 4.5 to about 7.0, and the molar ratio of daptomycin to the one or more basic amino acids or its salts (e.g., L-arginine or L-arginine HCl) is about 1:3.8 to about 1:5.6. In one embodiment, the molar ratio of daptomycin to the one or more basic amino acids or its salts (e.g., L-arginine or L-arginine HCl) is about 1:3.8 to about 1:5.5.

In one embodiment of any of the solid pharmaceutical compositions (e.g., solid lyophilized pharmaceutical compositions) described herein, the composition exhibits less than about 2.0% (by HPLC) increase of the sum of N-decanoyl-L-tryptophyl-D-asparaginyl-L-aspartyl-L-threonyne-glycyl-L-ornithyl-L-aspartyl-D-alanyl-L-aspartyl-glycyl-D-seryl-threo-3-methyl-L-glutamyl-3-anthraniloyl-L-alanine (Impurity A, lactone hydrolysis impurity) and N-decanoyl-L-tryptophyl-D-asparaginyl-L-aspartyl-2-aminobutenoyl-glycyl-L-ornithyl-L-aspartyl-D-alanyl-L-aspartyl-glycyl-D-seryl-threo-3-methyl-L-glutamyl-3-anthraniloyl-L-alanine (Impurity 1) upon storage from an initial point to 3 months at 40° C. and 75% relative humidity.

In one embodiment of any of the solid pharmaceutical compositions (e.g., solid lyophilized pharmaceutical compositions) described herein, the composition exhibits less than about 1.3% (by HPLC) increase of the sum of Impurity A and Impurity 1 upon storage from an initial point to 1 month at 40° C. and 75% relative humidity.

In one embodiment of any of the solid pharmaceutical compositions (e.g., solid lyophilized pharmaceutical compositions) described herein, the composition exhibits no more than about 1.5% (by HPLC) increase of the sum of Impurity A and Impurity 1 upon storage from an initial point to 12 months at 25° C. and 60% relative humidity.

In one embodiment of any of the solid pharmaceutical compositions (e.g., solid lyophilized pharmaceutical compositions) described herein, the composition exhibits no more than about 1.9% (by HPLC) increase in total impurities upon storage from an initial point to 1 month at 40° C. and 75% relative humidity.

Another embodiment of the present invention provides a solid pharmaceutical composition (e.g., a solid lyophilized pharmaceutical composition) consisting of daptomycin, one or more basic amino acids or its salts (e.g., L-arginine or L-arginine HCl), and one or more pH adjusting agents, wherein (i) the molar ratio of daptomycin to the one or more basic amino acids or its salts (e.g., L-arginine or L-arginine HCl) is from about 1:3.8 to about 1:5.6, (ii) the pH of the composition is about 4.5 to about 7 and (iii) the composition exhibits no more than about 4% (by HPLC) increase of total impurities from an initial time point to 6 months at 40° C./75% RH. In one embodiment, the molar ratio of daptomycin to the one or more basic amino acids or its salts (e.g., L-arginine or L-arginine HCl) is from about 1:3.8 to about 1:5.5.

Another embodiment of the present invention provides a solid pharmaceutical composition (e.g., a solid lyophilized pharmaceutical composition) consisting of daptomycin, one or more basic amino acids or its salt (e.g., L-arginine or L-arginine HCl), and one or more pH adjusting agents, wherein (i) the molar ratio of daptomycin to the one or more basic amino acids or its salts (e.g., L-arginine or L-arginine HCl) is from about 1:3.8 to about 1:5.6, (ii) the pH of the composition is about 4.5 to about 7 and (iii) the composition exhibits no more than about 3% (by HPLC) increase of total impurities from an initial time point to 3 months at 40° C./75% RH. In one embodiment, the molar ratio of daptomycin to the one or more basic amino acids or its salts (e.g., L-arginine or L-arginine HCl) is from about 1:3.8 to about 1:5.5.

Another embodiment of the present invention provides a solid pharmaceutical composition (e.g., a solid lyophilized pharmaceutical composition) consisting of (i) daptomycin, (ii) L-arginine hydrochloride, and (iii) 1N HCl and/or 2N NaOH.

Another embodiment of the present invention provides a stable pharmaceutical composition comprising daptomycin, one or more basic amino acids or its salts, and one or more pH adjusting agents, wherein (i) the composition is reconstituted using a pharmaceutically acceptable diluent and (ii) the reconstituted daptomycin composition is stable for about 10 days at refrigerated conditions (e.g., about 2° C. to about 8° C.).

Another embodiment of the present invention provides a solid pharmaceutical composition comprising daptomycin, wherein the solid pharmaceutical composition is prepared by a process comprising:
i) adding approximately 20-80% of a batch volume of solvent to a mixing vessel (at, e.g., 20-25° C.);
ii) optionally adding one or more stabilizers to the solvent (at, e.g., 20-25° C.) and stirring until complete dissolution;
iii) adjusting the pH of the solution to a targeted value using one or more pH adjusting agents;
iv) sprinkling (or adding) daptomycin in multiple portions (e.g., each portion containing about 1 to 10 kgs of daptomycin) to the solvent or excipient solution obtained in step
(iii) (e.g., over 30-180 minutes, such as 60-90 minutes, at 2-8° C.);
v) sprinkling (or adding) aqueous solvent to any foam generated during and after daptomycin addition and stirring until the foam is completely dissolved;
vi) optionally, adjusting the pH of the solution to a targeted pH after the addition of each portion of daptomycin to about 4.5 to about 7;
vii) adding additional solvent to provide a desired final volume; viii) filtering the solution (e.g., using a 0.2 μm filter);
ix) filling the solution is into vials and partially stoppering the vials; and
x) subjecting the filled partially stoppered vials to lyophilization in a freeze dryer,
xi) completely stoppering the vials, unloading, and sealing.

Another embodiment of the present invention provides a solid pharmaceutical composition (e.g., a solid lyophilized pharmaceutical composition) comprising daptomycin, one or more basic amino acids (e.g., L-arginine or a salt thereof (e.g., L-arginine HCl), and one or more pH adjusting agents wherein the composition is reconstituted in a suitable pharmaceutical diluent in less than about fifteen minutes (such as less than about 10 minutes or less than about 5 minutes). In one embodiment, the pharmaceutical composition consists of daptomycin, one basic amino acid (e.g., L-arginine HCl), a solvent, and one or more pH adjusting agents.

### DETAILED DESCRIPTION OF THE INVENTION

The present inventors have invented daptomycin lyophilized pharmaceutical compositions having enhanced stability and reduced reconstitution time.

The present inventors have surprisingly found that a significant daptomycin degradation-based impurity is N-decanoyl-L-tryptophyl-D-asparaginyl-L-aspartyl-2-aminobutenoyl-glycyl-L-ornithyl-L-aspartyl-D-alanyl-L-aspartyl-glycyl-D-seryl-threo-3-methyl-L-glutamyl-3-anthraniloyl-L-alanine, referred to herein as Impurity 1. The present invention relates, in one aspect, to methods of controlling the formation of Impurity 1, and to compositions containing acceptable amounts of Impurity 1 suitable for parenteral administration.

### Definitions

As used herein, “lyophilization” and “lyophilizing” means a stabilizing process used to remove a solvent from a pharmaceutical formulation; at low temperatures through sublimation (primary drying) and then desorption (secondary drying). A lyophilized formulation can be reconstituted in a simple manner to give a ready-to-use solution which contains no visible particles by addition of a solvent.

As used here, “batch” or “pharmaceutical batch” refers to material produced by a single execution of a compounding process of various embodiments of the present invention.

“Batches' or “pharmaceutical batches' as defined herein may include a single batch, wherein the single batch is representative of all commercial batches.

The term “stable” when used in connection with a reconstituted solution refers to a solution in which total impurities in the solution are less than 5% after storage for 18 hours at room temperature.

### Lyophilized Daptomycin Formulations

In certain embodiments, the present disclosure provides solid daptomycin formulations containing a stabilizer selected from a basic amino acid selected from arginine, histidine, lysine and pharmaceutically acceptable salts of any of the foregoing, the formulations having reconstitution times generally less than about 15 minutes in a pharmaceutically acceptable diluent. For example, a solid daptomycin composition (e.g., 500 mg of a solid daptomycin composition) prepared by lyophilization of a daptomycin solution comprising one or more basic amino acids selected from arginine, histidine, lysine, and pharmaceutically acceptable salts of any of the foregoing, or a combination thereof, can be reconstituted in 0.9% aqueous sodium chloride, sterile water for injection or bacteriostatic water for injection at room temperature (e.g., about 25° C.) in about 15 minutes or less (including reconstitution times of about 10 minutes or less, about 7 minutes or less, about 5 minutes or less, about 3 minutes or less and about 1 minute or less).

Reconstitution times can be determined by injecting from about 0.5 mL to about 20 mL of a pharmaceutically acceptable diluent into a vial containing from about 10 mg to about 1000 mg of lyophilized daptomycin composition. The resultant mixture may be swirled for about one minute and allowed to stand. The reconstitution time is the time required from addition of the diluent to the solid daptomycin composition to complete dissolution of the solid daptomycin composition in the diluent.

Solid daptomycin compositions having a faster reconstitution time as described in any embodiment herein are obtainable from aqueous daptomycin solutions at a suitable pH (e.g., from about 4.5 to about 7) and temperature (e.g., from about 5° C. to about 40° C.). In general, the solid daptomycin compositions can be made from an aqueous solution of daptomycin at a pH above the isoelectric point of daptomycin.

In one embodiment, a solid composition of the present invention comprises daptomycin, L-arginine or a pharmaceutically acceptable salt thereof (e.g., L-arginine HCl), and one or more pH adjusting agents.

In one embodiment, any solid composition of daptomycin described herein is free or substantially free of a preservative.

In another embodiment, a solid composition of the present invention consists essentially of (or consists of) daptomycin, one or more basic amino acids (e.g., L-arginine or its pharmaceutically acceptable salt (e.g., L-arginine HCl or L-arginine phosphate), and one or more pH adjusting agents.

In some embodiments, the molar ratio of daptomycin to one or more basic amino acids is from about 1:3.5 to about 1:7. In some embodiments, the amount of daptomycin in the lyophilized composition is about 100 mg to about 1000 mg or about 350 mg or about 500 mg. The quantity of the one or more basic amino acids (e.g., L-arginine or its salt, such as L-arginine HCl) in the lyophilized composition is about 50 mg to about 2500 mg.

In one embodiment, the solid pharmaceutical composition comprises (i) about 350 mg daptomycin, (ii) about 210 mg L-arginine HCl, (iii) optionally sodium hydroxide and/or hydrochloric acid as pH adjusting agents and (iv) optionally a buffering agent. In one embodiment, the composition is free of a preservative.

In another embodiment, the solid pharmaceutical composition comprises (i) about 500 mg daptomycin, (ii) about 300 mg L-arginine HCl, (iii) optionally sodium hydroxide and/or hydrochloric acid as pH adjusting agents and, optionally, a buffering agent. In one embodiment, any solid composition of daptomycin described herein is free or substantially free of a preservative.

In one embodiment, the pH of the lyophilized pharmaceutical composition is from about 4.5 to about 7, such as from about 5 to about 6, or about 5.25.

In certain aspects, the solid daptomycin compositions according to any of the embodiments described herein have a pH from about 4.5 to about 7, and the composition exhibits no more than about 1.5% (by HPLC) increase of Impurity 1 and no more than about 3% (by HPLC) increase of total impurities from an initial time point to 3 months or 6 months at 40° C. and 75% relative humidity (RH). In another embodiment, the composition exhibits no more than about 1.5% (by HPLC) increase of Impurity 1 and no more than about 3% (by HPLC) increase of total impurities from an initial time point to 12 months, 18 months, or 24 months at 40° C. and 75% relative humidity (RH).

In certain aspects, the solid daptomycin compositions according to any of the embodiments described herein have a pH from about 5 to about 6, or about 5.25, and the composition exhibits no more than about 0.75% (by HPLC) increase of Impurity 1 from an initial time point to 3 months or 6 months at 40° C. and 75% RH. In another embodiment, the composition exhibits no more than about 0.75% (by HPLC) increase of Impurity 1 from an initial time point to 12 months, 18 months, or 24 months at 40° C. and 75% RH. In another embodiment, the composition exhibits no more than about 0.75% (by HPLC) increase of Impurity 1 from an initial time point to 12 months, 18 months, or 24 months at 40° C. and 75% relative humidity (RH).

In certain aspects, the solid daptomycin compositions according to any of the embodiments described herein have a pH from about 5 to about 6, or about 5.25, and the composition exhibits no more than about 1% (by HPLC) increase of total impurities from an initial time point to 3 months or 6 months at 40° C. and 75% RH. In another embodiment, the composition exhibits no more than about 1% (by HPLC) increase of total impurities from an initial time point to 12 months, 18 months, or 24 months at 40° C. and 75% relative humidity (RH).

In certain aspects, the solid daptomycin compositions according to any of the embodiments described herein have a pH from about 5 to about 6, or about 5.25, and the composition exhibits no more than about 1% (by HPLC) increase of total impurities from an initial time point to 3 months, 6 months, 12 months, 18 months or 24 months at 25° C. and 60% RH.

In one embodiment, the solid daptomycin composition has a pH from about 5 to about 6, preferably about 5.25, and the composition exhibits no more than about 1.5% (by HPLC) increase of Impurity 1 from an initial time point to 3 months, 6 months, 12 months, 18 months or 24 months at 25° C. and 60% RH.

### Stabilizers

Formulations including daptomycin may also include at least one stabilizer. Stabilizers which may be used in any of the embodiments described herein include, but are not limited to, basic amino acids, including, e.g., arginine (e.g., L-arginine), histidine (e.g., L-histidine), and lysine (e.g., L-lysine) or a pharmaceutically acceptable salt of any of the foregoing, polymers including, e.g., polyvinylpyrrolidone (PVP), including PVP K12 and alcohols including, e.g., tertiary-butyl alcohol. Combinations of these stabilizers can also be included in any of the embodiments disclosed herein. In some embodiments, the stabilizer may be a basic amino acid. In some further embodiments, the stabilizer may be arginine, or a pharmaceutically acceptable salt thereof. In some embodiments, the stabilizer may be L-arginine or its hydrochloride or phosphate salt (L-arginine HCl or L-arginine phosphate).

In one embodiment, any pharmaceutical composition described herein is free or substantially free (e.g., contains less than 0.5, 0.2, 0.1, 0.05, 0.02, or 0.01% w/w) of (a) any amino acid other than L-arginine or a salt thereof (such as L-histidine, L-isoleucine, or both), (b) calcium chloride, or (c) any combination of any of the foregoing.

In additional embodiments, any of the compositions described herein do not contain (i.e., are free of, or substantially free of (e.g., contain less than about 0.5% w/w), of stabilizers (e.g., glycine), saccharides, including monosaccharides and disaccharides, non-reducing sugars (e.g., sucrose, maltose, fructose, dextrose, trehalose, lactose), and alcohol sugars (e.g., mannitol, erythritol, glycerol, lactitol, isomaltose, maltitol, sorbitol and xylitol).

The amounts and concentrations of stabilizers provided in the compositions described herein, and the particular combinations thereof are preferably selected to stabilize the solid daptomycin composition stored at various storage conditions, for example 2-8° C., room temperature (e.g., about 25° C.) or accelerated conditions (e.g., 45° C./75% RH) for a period of at least 3 months, at least 6 months, at least 12 months, at least 18 months, at least 24 months or at least 36 months. The amounts and concentrations of stabilizers provided in the compositions according to the various embodiments of the present invention also stabilize the reconstituted solution and diluted solution obtained using the solid daptomycin compositions described herein for sufficient time at various storage conditions for administration to a patient in need thereof. The molar ratio of daptomycin to stabilizer can be selected to obtain liquid compositions, which, if converted to solid compositions, exhibit favorably rapid reconstitution times in the aqueous solvents in which they are reconstituted. For example, a daptomycin composition of the present invention may contain daptomycin and stabilizer (e.g., L-arginine HCl) in a molar ratio of about 1:3.5 to about 1:7, e.g., about 1:3.8 to about 1:5.6, or about 1:3.8 to about 1:5.5.

The molar ratio of daptomycin to L-arginine hydrochloride may play a significant role in the stability profile of daptomycin composition. For example, a daptomycin composition containing L-arginine HCl in a molar ratio of 1:0.6, 1:0.9 and 1:1.2 exhibits not less than about 5.5% (by HPLC) of total impurities upon storage for 1 month at 40° C./75% RH.

A solid daptomycin composition (pH 7) containing about 1:3.8 molar ratio of daptomycin to arginine hydrochloride exhibits about 4.25% (by HPLC) increase of total impurities from an initial time point to 6 months at 40° C./75% RH whereas the composition having about 1:4.6 molar ratio of daptomycin to L-arginine HCl exhibits about 2.25% (by HPLC) increase of total impurities under the same conditions. A composition (pH 6.8) containing about a 1:11.5 molar ratio of daptomycin (350 mg) to L-arginine HCl (525 mg) exhibits about 2% (by HPLC) increase of total impurities from an initial time point to 1 month at 40° C./75% RH (i.e., after storage for 1 month at 40° C./75% RH).

Surprisingly, a solid composition containing about 1:4.6 molar ratio of daptomycin to L-arginine HCl results in a stable daptomycin composition. Furthermore, a solid composition containing about 1:4.6 molar ratio of daptomycin to L-arginine HCl at a pH of about 5 to about 6 results in a composition exhibiting enhanced stability.

The present invention further provides a solid pharmaceutical composition comprising about 350 mg daptomycin, about 210 mg L-arginine HCl, hydrochloric acid and/or sodium hydroxide as pH adjusting agents and, optionally, a buffering agent, wherein (i) the pH of the composition is about 4.5 to about 7, and (ii) the composition exhibits no more than about 3% (by HPLC) increase of total impurities and no more than about 1.5% (by HPLC) increase of Impurity 1 from an initial time point to 3 months at 40° C./75% RH.

The present invention further provides a solid pharmaceutical composition comprising about 500 mg daptomycin, about 300 mg L-arginine HCl, hydrochloric acid and/or sodium hydroxide as pH adjusting agents and, optionally, a buffering agent wherein (i) the pH of the composition is about 4.5 to about 7 and (ii) the composition exhibits no more than about 2% (by HPLC) increase of total impurities from an initial time point to 24 months at 25° C./60% RH.

The present invention further provides a solid pharmaceutical composition comprising about 500 mg daptomycin, about 300 mg L-arginine HCl, hydrochloric acid and/or sodium hydroxide as pH adjusting agents and, optionally, a buffering agent wherein (i) the pH of the composition is about 5 to about 6 and (ii) the composition exhibits no more than about 1% (by HPLC) increase of total impurities from an initial time point to 24 months at 25° C./60% RH.

Structures of some of the daptomycin impurities are shown below in Table 1. The inventors have identified a new impurity (Impurity 1) as a significant degradation impurity of lyophilized daptomycin formulations. The present invention therefore relates, in certain embodiments, to methods of controlling this impurity in such formulations. Impurity 1 is formed by a combination of ester hydrolysis (lactone hydrolysis) and dehydration at the threonine residue.

TABLE 1
Impurities of Daptomycin

| Impurity | Chemical | |
|---|---|---|
| Name | Name | Chemical Structure |
| Lactone hydrolysis (Impurity A) | N-decanoyl-L- tryptophyl-D- asparaginyl-L- aspartyl- L-threonyne- glycyl-L- ornithyl-L- aspartyl-D- alanyl-L- aspartyl- glycyl-D- seryl-threo-3- methyl-L- glutamyl-3- anthraniloyl- L-alanine | 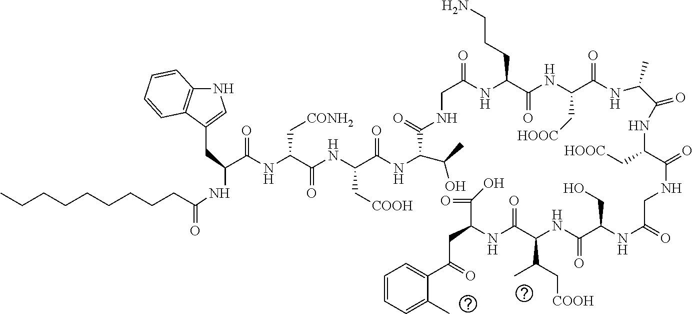 [SMILES: CCCCCCCCCC(=O)N[C@@H](Cc1c[nH]c2ccccc12)C(=O)N[C@H](CC(N)=O)C(=O)N[C@@H](CC(=O)O)C(=O)N[C@H](C(=O)NCC(=O)N[C@@H](CCCN)C(=O)N[C@@H](CC(=O)O)C(=O)N[C@H](C)C(=O)N[C@@H](CC(=O)O)C(=O)NCC(=O)N[C@H](CO)C(=O)N[C@H](C(=O)N[C@@H](CC(=O)c1ccccc1C)C(=O)O)C(C)CC(=O)O)[C@@H](C)O] |
| Impurity 1 | N-decanoyl-L- tryptophyl-D- asparaginyl-L- aspartyl- 2-amino- butenoyl- glycyl-L- ornithyl-L- aspartyl-D- alanyl- L- aspartyl- glycyl-D- seryl-threo-3- methyl-L- glutamyl-3- anthraniloyl- L-alanine | 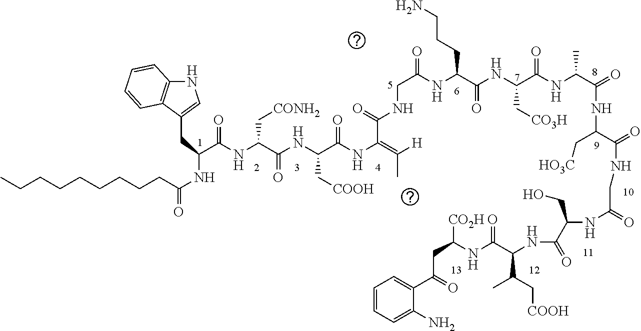 |
| β-Isomer Dapto- mycin | N-decanoyl-L- tryptophyl-D- asparaginyl-L- aspartyl- L-threonyl- glycyl-L- ornithyl-L- aspartyl-D- alanyl-L-β- aspartyl- glycyl-D- seryl-threo-3- methyl-D- glutamyl-3- anthraniloyl- L-alanine ε1-lactone | 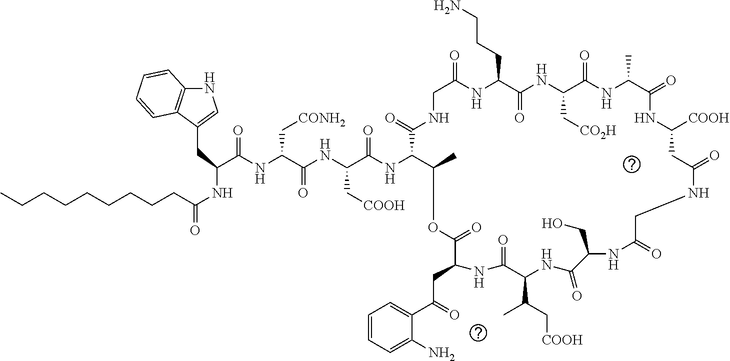 [SMILES: CCCCCCCCCC(=O)N[C@@H](Cc1c[nH]c2ccccc12)C(=O)N[C@H](CC(N)=O)C(=O)N[C@@H](CC(=O)O)C(=O)N[C@@H]1C(=O)NCC(=O)N[C@@H](CCCN)C(=O)N[C@@H](CC(=O)O)C(=O)N[C@H](C)C(=O)N[C@H](C(=O)O)CC(=O)NCC(=O)N[C@H](CO)C(=O)N[C@@H](C(C)CC(=O)O)C(=O)N[C@@H](CC(=O)c2ccccc2N)C(=O)O[C@@H]1C] |
| Anhydro- dapto- mycin | N-decanoyl-L- tryptophyl-D- asparaginyl-L- aspartyl- L-threonyl- glycyl-L- ornithyl-L- aspartyl-D- alanyl-L- succin- immidil- glycyl- D-seryl-threo- 3-methyl-L- glutamyl-3- anthraniloyl- L-alanine ε1-lactone | 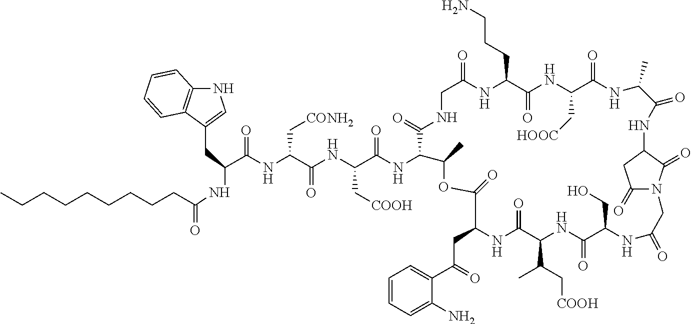 [SMILES: CCCCCCCCCC(=O)N[C@@H](Cc1c[nH]c2ccccc12)C(=O)N[C@H](CC(N)=O)C(=O)N[C@@H](CC(=O)O)C(=O)N[C@@H]1C(=O)NCC(=O)N[C@@H](CCCN)C(=O)N[C@@H](CC(=O)O)C(=O)N[C@H](C)C(=O)NC2CC(=O)N(CC(=O)N[C@H](CO)C(=O)N[C@@H](C(C)CC(=O)O)C(=O)N[C@@H](CC(=O)c3ccccc3N)C(=O)O[C@@H]1C)C2=O] |

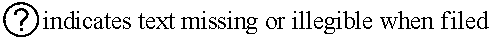

### pH Adjusting Agents

The pH of the daptomycin compositions disclosed herein may be adjusted, e.g., to about 4.5 to about 7. For example, solid daptomycin compositions may be obtained from bulk solutions having a pH of about 4.5 to about 7. The pH of the daptomycin bulk solution can be adjusted to about 4.5, about 5, about 5.25, about 5.5, about 5.75, about 6, about 6.5 or about 7 prior to conversion to a solid form (lyophilization). The pH adjusting agent may be selected from bases including, but not limited to, sodium hydroxide, potassium hydroxide, and calcium hydroxide, and acids such as, but not limited to, hydrochloric acid, sulfuric acid, and phosphoric acid. The basic amino acids, including e.g., L-arginine, L-lysine and their salts, can also act as a pH adjusting agents. The inventors have surprisingly found that the concentration of the pH adjusting agent used in the preparation of the solid daptomycin composition and the pH of the bulk solution controls the formation of one or more impurities during the manufacturing of lyophilized daptomycin compositions.

### Buffering Agents

The aqueous daptomycin compositions described herein may optionally include a buffering agent, to stabilize the composition at a desired pH, for example, a pH from about 4.5 to about 7. Buffering agents may include, for example, phosphate, citrate, maleate, or carbonate buffers, or any combination thereof, along with any pharmaceutically acceptable counterions. The concentration of the buffering agent can be varied, based on the molar ratio of buffering agent to daptomycin. The buffering agent may be added in anhydrous or hydrate form. Specific examples of buffering agents are sodium or potassium salts of phosphoric acid. The basic amino acids, including, e.g., arginine, lysine and the like (and their salts) can also act as a buffering agent.

### Compounding Process

The solid pharmaceutical compositions according to the present invention may be prepared by providing a liquid solution captaining daptomycin, one or more stabilizers and one or more pharmaceutically acceptable excipients. The solvent used in the preparation of the liquid solution may be selected from water for injection (WFI), an alcohol (such as tertiary butanol) or a combination thereof. The preparation process involves initial addition of one or more stabilizers and optionally other excipient(s), for example pH adjusting agent, to the solvent followed by addition of daptomycin or vice versa under stirring. Daptomycin and stabilizers can also be added simultaneously to the solvent to obtain a solution. The pH of the bulk solution may be adjusted to about 4.5 to about 7 after completing the addition of all the excipients.

Another embodiment of the present invention relates to a large-scale manufacturing process for preparing a pharmaceutical composition comprising daptomycin, e.g., for use as an antibacterial agent in a subject in need thereof, the process comprising:
i) adding approximately 20-80% by volume of a batch volume of solvent to a mixing vessel (at, e.g., 20-25° C.);
ii) optionally adding one or more stabilizers to the solvent (at, e.g., 20-25° C.) and stirring until complete dissolution;
iii) adjusting the pH of the solution to a targeted value using one or more pH adjusting agents;
iv) adding daptomycin in multiple portions (e.g., each portion containing about 1 to 10 kgs of daptomycin) to the solvent or excipient solution obtained in step (iii) (e.g., over 30-180 minutes (e.g., 60-90 minutes) at 2-8° C.);
v) sprinkling, or adding, aqueous solvent to any foam generated during and after daptomycin addition and stirring until the foam is completely dissolved;
vi) optionally, adjusting the pH of the solution to a targeted pH after the addition of each portion of daptomycin to about 4.5 to about 7;
vii) adding additional solvent to provide a desired final volume;
viii) filtering the solution (e.g., using a 0.2 μm filter);
ix) filling the solution is into vials and partially stoppering the vials; and
x) subjecting the filled partially stoppered vials to lyophilization in a freeze dryer; and
xi) completely stoppering the vials, unloading, and sealing.

The inventors have surprisingly found that the addition of daptomycin in multiple portions results in better dissolution of daptomycin and suppressing foam formation during dissolution of the daptomycin as well as reducing the levels of impurities during the compounding process. In one embodiment, each portion of daptomycin is in the range of about 1 to 10 kg, e.g., about 2 to about 6 kg (e.g., for a batch size of about 30 liters to about 500 liters).

Daptomycin is completely dissolved by efficiently mixing one or more solvents containing daptomycin. Efficient mixing is achieved by using one or more mixing devices. In certain embodiments, the mixing device is a paddle mixer, magnetic stirrer, shaker, re-circulating pump, homogenizer, or any combination thereof. In some embodiments, the mixing device is a homogenizer, a paddle mixer, or a combination thereof. The mixing device may be applied at a mixing rate of about 50 to about 2500 RPM, e.g., about 150 to about 1000 RPM.

Mixing the aqueous solvent optionally containing one or more excipients and daptomycin may be performed at, e.g., about 2-25° C. for about 30 to about 180 minutes (e.g., about 60 to about 90 minutes) at about 50 to about 2000 rpm. Upon complete dissolution of daptomycin in the aqueous solvent, the mixing speed may be reduced to about 25 to about 300 RPM.

Conventional processes for the preparation of daptomycin bulk solutions generate foam during daptomycin addition and dissolution. Foam generated during the addition of daptomycin entraps the daptomycin; therefore, wetting of daptomycin is minimized. The present inventors have surprisingly identified a new process for the preparation of daptomycin compositions which suppresses foam generation during daptomycin addition and dissolution. In one embodiment, the process comprises adding (e.g., sprinkling) water for injection on top of the foam generated during the compounding process.

The pH of the bulk solution may be adjusted to about 4.5 to about 7 by efficiently mixing in the pH adjusting agent including, but not limited to, HCl and/or NaOH. The pH adjusting agent may be mixed with the daptomycin solution optionally containing one or more excipients by using one or more mixing devices.

Upon complete dissolution of all the ingredients, the bulk solution may be made up to 100% of the batch volume (desired final volume) using a solvent. The bulk solution may be filtered through a filter (such as a 0.2 micron filter) and subjected to lyophilization, spray drying or fluid-bed drying to obtain a solid daptomycin composition according to any of the embodiments described herein.

### Solid Daptomycin Manufacturing Processes

Solid pharmaceutical compositions may be prepared by any suitable method, including lyophilization, spray-drying or fluid bed drying. The resulting solid daptomycin composition can be a lyophilized, freeze-dried, spray-dried, fluid bed-dried, spray congealed, precipitated or crystallized powder or amorphous solid. In certain embodiments, the solid daptomycin composition is a lyophilized or spray-dried powder.

The present invention provides methods for preparing lyophilized daptomycin formulations. The daptomycin formulations can be lyophilized from suitable solvents, including, for example, water, alcohols (such as tertiary butanol, aqueous butanol, aqueous ethanol), or any combination thereof. Processes which may be utilized to prepare the lyophilized daptomycin compositions disclosed herein are known to those of ordinary skill in the art using appropriate equipment typically used in the art. Exemplary lyophilization processes include those described in “Lyophilization: Introduction and Basic Principles,” by Thomas A. Jennings, InterPharm Press, 1999. In some embodiments, the processes described herein include forming an aqueous solution of daptomycin and at least one additive, adjusting the solution pH to about 4.5 to about 7.0, and lyophilizing the pH-adjusted solution to form a lyophilizate. In one embodiment, the process includes: (1) forming an aqueous solution of daptomycin at a pH of about 4.0 to about 5.0; (2) dissolving an additive as described herein in the aqueous solution or vice-versa; (3) adjusting the pH to about 4.5 to about 7.0; and (4) lyophilizing the solution to obtain a solid composition. In other embodiments, the process includes: (1) forming an aqueous tertiary-butanol solution of one or more additives as described herein at a pH of about 4.5 to about 6.0; (2) dissolving daptomycin in the solution; (3) adjusting the pH to about 4.5 to about 7; and (4) lyophilizing the solution to obtain a solid composition.

The lyophilization process may involve freezing, primary drying and secondary drying. Each of these steps includes individual parameters of temperature, ramp time, soak time and vacuum setting, readily known to one of ordinary skill in the art.

### Reconstitution and Dilution of Lyophilized Daptomycin Compositions

The solid daptomycin compositions according to any of the embodiments described herein may be reconstituted in a suitable liquid diluent. The addition of liquid diluent may be performed over a period of a few seconds, e.g., about 1-90 seconds, or about 10-30 seconds. The diluent may be added along the wall of the container (e.g., vial) or directly to the lyophilized composition. Upon completion of the addition of the diluent, the container may be gently rotated or swirled for a few minutes, e.g., for less than five minutes, until fully dissolved. The liquid diluent for reconstitution may be selected from water for injection (WFI), 0.9% sodium chloride, bacteriostatic water for injection, Lactated Ringer's injection, or any other pharmaceutically acceptable liquid diluent known in the art. The solid daptomycin compositions according to the embodiments described herein generate little or no foam during the reconstitution process. The daptomycin concentration in the reconstituted solution is about 5 mg/mL to about 100 mg/mL, e.g., about 75 mg/mL, or about 50 mg/mL. The reconstituted solution may be stored in a vial or syringe (for example, a polypropylene syringe).

The reconstituted solution obtained from WFI is stable for about 60 hours, or about 48 hours, or about 24 hours at room temperature (25° C.). The reconstituted solution obtained from WFI is stable for about 120 hours, or about 96 hours, or about 84 hours, or about 72 hours at 2-8° C.

The reconstituted solution obtained from bacteriostatic WFI is stable for about 60 hours, or about 48 hours, or about 24 hours at room temperature (25° C.). The reconstituted solution obtained from bacteriostatic WFI is stable for about 168 hours, or about 144 hours, or about 120 hours at 2-8° C.

The reconstituted solution may be diluted using a diluent such as sterile WFI, bacteriostatic water for injection, 0.9% sodium chloride, lactated Ringer's solution or any other pharmaceutically acceptable diluents to obtain a diluted daptomycin having a concentration in the range of about 1 mg/ml to about 75 mg/ml for parenteral administration including, e.g., IV push, IV infusion or SC administration.

The reconstituted daptomycin solution obtained using sterile WFI, bacteriostatic WFI, 0.9% saline, or any other pharmaceutically acceptable diluent may be diluted using sterile WFI, bacteriostatic WFI, 0.9% saline or lactated Ringer's solution or any other pharmaceutically acceptable diluent to obtain diluted daptomycin in the concentration range of about 1 mg/ml to about 75 mg/ml, e.g., about 3 mg/ml to about 10 mg/ml.

In one aspect, the present invention relates to a solid pharmaceutical composition containing daptomycin and L-arginine HCl (e.g., in a molar ratio of about 1:4.6) reconstituted with sterile water for injection or bacteriostatic water for injection and diluted with 0.9% NaCl wherein the composition is stable for about 1.5 days, or about 1 day at room temperature (RT) and 10 days under refrigerated conditions (e.g., at about 2-8° C.).

In one aspect, the present invention relates to a solid pharmaceutical composition comprising daptomycin and L-arginine HCl (e.g., in a molar ratio of about 1:4.6) reconstituted with sterile water for injection or bacteriostatic water for injection and diluted with Lactated Ringers' injection, wherein the composition is stable for about 1 day at RT and about 2 days under refrigerated conditions (e.g., about at about 2-8° C.).

In one aspect, the present invention relates to a solid pharmaceutical composition comprising daptomycin and L-arginine HCl (e.g., in a molar ratio of about 1:4.6) reconstituted with sterile water for injection or 0.9% NaCl and diluted with 0.9% NaCl, wherein the composition is stable for about 18 hours at RT and about 10 days under refrigerated conditions (e.g., about at about 2-8° C.).

In one aspect, the present invention relates to a solid pharmaceutical composition comprising daptomycin and L-arginine HCl (e.g., in a molar ratio of about 1:4.6) reconstituted with sterile water for injection or 0.9% NaCl and diluted with Lactated Ringer's injection, wherein the composition is stable for 18 hours at RT and 2 days under refrigerated conditions (e.g., about at about 2-8° C.).

The diluted solutions according to any of the embodiments described herein may be stored in a vial, a syringe or a bag.

TABLE 2
In-use Stability of Claimed Daptomycin Composition
in Various Diluents and Containers

| | | In-Use Shelf-Life | |
|---|---|---|---|
| | | Room | |
| | | Temperature | Refrigerated |
| | | (20° C.-25° C., | (2° C.-8° C., |
| Container | Diluent | 68° F.-77° F.) | 36° F.-46° F.) |
| Vial | Sterile Water for Injection | 18 hours | 5 days |
| Syringe* | Sterile Water for Injection | 18 hours | 10 days |
| Intravenous | Vial reconstituted with Sterile Water for | 18 hours | 10 days |
| Bag | Injection or 0.9% saline and immediately | | |
| | diluted with 0.9% sodium chloride | | |
| | injection | | |
| | Vial reconstituted with Sterile Water for | 18 hours | 2 days |
| | Injection and immediately diluted with | | |
| | Ringer's injection | | |

*Polypropylene syringe with elastomeric plunger stopper

### Liquid Daptomycin Compositions

Formulations of daptomycin can be prepared as liquid pharmaceutical compositions, or as solid pharmaceutical compositions. Liquid pharmaceutical daptomycin compositions may be prepared by a process comprising: dissolving daptomycin in a solvent or a mixture of solvents, adding one more excipients such as a stabilizer, a buffering agent, etc., and adjusting the pH of the solution to about 4.5 to about 7. Alternatively, a liquid composition may be obtained by reconstitution of a solid daptomycin composition according to any of the embodiments of described herein in one or more liquid diluent.

In various embodiments, the liquid pharmaceutical compositions described herein comprise daptomycin, a basic amino acid or a salt thereof (e.g., L-arginine or L-arginine HCl), a pH adjusting agent, optionally on or more buffering agents, and a liquid diluent (such as water for injection, 0.9% sodium chloride or any other conventional diluent).

In another embodiment, the liquid pharmaceutical compositions described herein consist essentially of daptomycin, L-arginine (or its salt, such as L-arginine HCl), sodium hydroxide, optionally a buffering agent, and a liquid diluent selected from sterile WFI, bacteriostatic water for injection and 0.9% sodium chloride.

### Methods of Administration

In a further aspect, the present invention relates to parenteral administration of daptomycin (e.g., a daptomycin composition according to any of the embodiments described herein) to a patient in need thereof (e.g., for the treatment of a bacterial infection). In certain embodiments, the administration is intravenous (IV) or subcutaneous (SC) administration. In one embodiment, the present invention relates to intravenous administration which includes bolus intravenous and intravenous infusion. In another embodiment, the present invention relates to subcutaneous administration. Subcutaneous administration may involve a few seconds to a few minutes, for example, less than about 10 seconds, less than about 30 seconds, less than about 60 seconds, less than about 2 minutes, less than about 5 minutes, less than about 10 minutes, less than about 15 minutes or less than about 20 minutes. The subcutaneous administration volume may be in the range of less than about 25 ml, e.g., less than about 20 ml, less than about 15 ml, less than about 10 ml, less than about 5 ml, or less than about 3 ml. The subcutaneous administration may involve a prefilled syringe, a pump, a device or an on-body infuser.

### Methods of Treatment

Any of the daptomycin compositions described herein can be used therapeutically for the treatment of bacterial infections in a subject having need of such treatment. The methods of treatment can include administering to a subject in need thereof, a reconstituted daptomycin composition, made from an effective amount of a lyophilized daptomycin formulation including an additive selected from basic amino acids, according to any of the embodiments described herein.

In one embodiment, any of the daptomycin compositions described herein can be administered therapeutically for the treatment of skin and skin structure infections (cSSSI) caused by susceptible isolates of the following Gram-positive bacteria: Staphylococcus aureus (including methicillin-resistant isolates), Streptococcus pyogenes, Streptococcus agalactiae, Streptococcus dysgalactiae subsp. equisimilis, and Enterococcus faecalis (vancomycin-susceptible isolates only).

In one embodiment, any of the daptomycin compositions described herein can be administered therapeutically for the treatment of Staphylococcus aureus bloodstream infections (bacteremia) in adult patients, including those with right-sided infective endocarditis, caused by methicillin-susceptible and methicillin-resistant isolates.

In one embodiment, any of the daptomycin compositions described herein can be administered therapeutically for the treatment of pediatric patients (1 to 17 years of age) with Staphylococcus aureus bloodstream infections (bacteremia).

In one embodiment, any of the daptomycin compositions described herein is administered to a pediatric patient according to the following dosing schedule:
(a) for a patient 1 to less than 2 years of age, 10 mg/kg once every 24 hours infused over 60 minutes, for up to 14 days;
(b) for a patient 2 to 6 years of age, 9 mg/kg once every 24 hours infused over 60 minutes, for up to 14 days;
(c) for a patient 7 to 11 years of age, 7 mg/kg once every 24 hours infused over 30 minutes, for up to 14 days; and
(d) for a patient 12 to 17 years of age, 5 mg/kg once every 24 hours infused over 30 minutes, for up to 14 days.

In one embodiment, any of the daptomycin compositions described herein is administered to a patient (18 years or older) according to the following dosing schedule: 4 mg/kg once every 24 hours infused for 7 to 14 days.

### EXAMPLES

The present disclosure will be further understood by reference to the following non-limiting examples. The following examples are provided for illustrative purposes only and are not to be construed as limiting the scope of the invention in any manner.

### Example 1: Daptomycin Compositions Containing Various Stabilizers

The general manufacturing process was as follows:
a) optionally one or more stabilizers were added to a solvent at 50% by volume of the batch volume and stirred until complete dissolution. Further, the pH of the solution may be adjusted to the targeted value using one or more pH adjusting agent,
b) daptomycin was added to the solvent or excipient solution obtained in step (a) and stirred until complete dissolution at 2-8° C.,
c) optionally, a separate excipient solution containing one or more stabilizers and one or more pH adjusting agent was prepared by dissolving them in the targeted solvent,
d) the daptomycin solution obtained in step (b) was added to the excipient solution obtained in step (c),
e) optionally, the pH of the compounded solution was adjusted to a target pH,
f) the volume was made up to 100% of the batch volume using the solvent,
g) the final bulk solution was filtered through a 0.2 micron filter,
h) the sterile bulk solution was filled into the glass vials and partially stoppered, and
i) the partially stoppered vials were lyophilized in a freeze dryer, followed by complete stoppering, unloading and sealing.

The HPLC method (hereafter Method-1) disclosed in Table 3 of U.S. Pat. No. 8,835,382 (“the '382 Patent”) was initially used to analyze the daptomycin composition prepared according to the general manufacturing process described above.

Formulations containing 350 mg daptomycin and different stabilizers were prepared according to the general manufacturing process and provided in Table 3 below. Water for injection was used as the solvent in all the formulations and the pH of these formulations was adjusted to 7 using one or more of the specified pH adjusting agent(s). These formulations were tested for stability under various conditions, including accelerated stability condition (40±2° C./70±5% RH), and analyzed using HPLC Method-1.

TABLE 3
Daptomycin Formulations 350 mg/Vial
Containing Various Stabilizers

| S. | | Quantity | |
|---|---|---|---|
| No | Ingredient | (mg/vial) | pH Adjusting Agent(s) |
| 1 | Polyvinyl Pyrrolidone | 50 | 5N NaOH & 1N HCl |
| | (PVP) K-12 | | |
| 2 | L-Lysine HCl | 140 | 5N NaOH & 1N HCl |
| 3 | Sucrose | 499 | 5N NaOH |
| 4 | L-Arginine HCl | 175 | 5N NaOH & 1N HCl |

The inventors analyzed the daptomycin formulations containing different stabilizers and surprisingly found that the stabilizer L-arginine HCl provided a stable daptomycin formulation. Stability data under the accelerated conditions (40° C./75 RH) for up to 6 months and long-term storage conditions (25° C./60% RH) are shown in Table 4. Surprisingly, the L-arginine HCl salt containing formulation resulted in a much lower level of total impurity and sum of lactone hydrolysis impurity (Impurity A) plus Impurity 1, when compared to another amino acid (L-lysine HCl) containing formulation.

TABLE 4
Stability Data of Daptomycin Compositions at Accelerated Conditions (40°
C./75% RH) and Room Temperature Conditions 25° C./60% RH)

| | | Related Substances | | | | Δ |
|---|---|---|---|---|---|---|
| | | “Lactone | Anhydro | Beta- | Total | (Total |
| Time period | Assay | hydrolysis” | Daptomycin | Isomer | Impurities | impurities) |
| (Months, M) | (in %) | peak* (%) | (%) | (%) | (%) | (%) |

Stabilizer: PVP K-12
a) 40° C./75% RH

| Initial | | 100.5 | 4.17 | 0.32 | 0.30 | 6.20 | — |
|---|---|---|---|---|---|---|---|
| 1 | M | 96.6 | 7.27 | 0.7 | 0.21 | 9.78 | 3.58 |
| 3 | M | 93.6 | 10.02 | 1.05 | 0.46 | 13.76 | 7.56 |
| 6 | M | 92.9 | 11.58 | 1.23 | 0.57 | 16.01 | 9.81 |

b) 25° C./60% RH

| | | | | | | | |
|---|---|---|---|---|---|---|---|
| 6 | M | 97.4 | 6.5 | 0.63 | 0.28 | 9.06 | 2.86 |
| 18 | M | 97.1 | 8.46 | 0.83 | 0.24 | 11.45 | 5.25 |

Stabilizer: L-Lysine HCl
a) 40° C./75% RH

| Initial | | 103.2 | 1.75 | 0.54 | 0.19 | 4.28 | — |
|---|---|---|---|---|---|---|---|
| 1 | M | 101 | 4.06 | 0.77 | 0.22 | 6.58 | 2.3 |
| 3 | M | 95.8 | 6.66 | 1.00 | 0.33 | 9.87 | 5.59 |
| 6 | M | 97.7 | 7.73 | 1.30 | 0.37 | 11.75 | 7.47 |

b) 25° C./60% RH

| | | | | | | | |
|---|---|---|---|---|---|---|---|
| 6 | M | 100.5 | 3.22 | 0.77 | 0.23 | 6.12 | 1.84 |
| 18 | M | 100.5 | 4.35 | 0.9 | 0.13 | 7.36 | 3.08 |

Stabilizer: Sucrose
a) 40° C./75% RH

| Initial | | 104.2 | 2.67 | 0.26 | 0.21 | 4.42 | — |
|---|---|---|---|---|---|---|---|
| 1 | M | 101.7 | 3.39 | 0.42 | 0.30 | 5.52 | 1.1 |
| 3 | M | 99.2 | 4.23 | 0.68 | 0.58 | 6.89 | 2.47 |
| 6 | M | 99.7 | 5.13 | 0.86 | 0.72 | 8.31 | 3.89 |

b) 25° C./60% RH

| | | | | | | | |
|---|---|---|---|---|---|---|---|
| 6 | M | 102.7 | 3.13 | 0.4 | 0.3 | 5.27 | 0.85 |
| 18 | M | 102.7 | 3.48 | 0.47 | 0.19 | 5.82 | 1.40 |

Stabilizer: L-Arginine HCl
a) 40° C./75% RH

| Initial | | 99.6 | 0.48 | 0.52 | 0.20 | 2.92 | — |
|---|---|---|---|---|---|---|---|
| 1 | M | 100.4 | 1.46 | 0.74 | 0.22 | 3.82 | 0.9 |
| 3 | M | 97.2 | 2.54 | 0.76 | 0.25 | 5.10 | 2.18 |
| 6 | M | 97.7 | 4.1 | 1.00 | 0.3 | 7.26 | 4.34 |

b) 25° C./60% RH

| | | | | | | | |
|---|---|---|---|---|---|---|---|
| 6 | M | 100.3 | 1.18 | 0.70 | 0.26 | 3.85 | 0.93 |
| 18 | M | 99.9 | 1.69 | 0.74 | 0.13 | 4.25 | 1.33 |

*HPLC Method-1 was subsequently determined to result in the co-elution of the lactone hydrolysis impurity (Impurity A) and Impurity-1, therefore the sum of these two impurities is reported as the “lactone impurity” in this table; see Example 5 for further details.

### Example 2: Daptomycin Composition Containing L-Arginine and a Bulking Agent

Daptomycin formulations, as described in Table 5, containing L-arginine HCl and bulking agent (mannitol) were prepared as per the general manufacturing process by dissolving the ingredient in water for injection and adjusting the pH to 6.8 followed by lyophilization.

TABLE 5
Daptomycin Formulations Containing L-Arginine HCl and Mannitol

| | L-Arginine | | Molar Ratio* | | pH | |
|---|---|---|---|---|---|---|
| | HCl | Mannitol | (Dap:L-Arg | | adjusting | |
| Batch No | (mg/vial) | (mg/vial) | HCl) | pH | Agent | Solvent |
| A (Supplier #1) | 42 | 210 | 1:0.9 | 6.8 | 5N NaOH | WFI |
| B (Supplier #2) | 42 | 210 | 1:0.9 | 6.8 | 5N NaOH | WFI |

It should be noted that the formulations containing mannitol as one of the stabilizers resulted in an unacceptably high initial level of impurities, primarily the “lactone hydrolysis” peak, as evident from Table 6.

TABLE 6
Stability Study of Daptomycin Formulations Containing L-Arginine
HCl and Mannitol at Accelerated Conditions (40° C./75% RH)

| | | | | | | |
|---|---|---|---|---|---|---|
| | | “Lactone | Anhydro | Beta- | Total | Δ (Total |
| Time Point | Assay | hydrolysis” | daptomycin | Isomer | Impurities | Impurities) |
| (Month, M) | (%) | Peak^{#} (%) | (%) | (%) | (%) | (%) |

Batch A; Supplier #1; Daptomycin: 350 mg; L-arginine HCl: 42 mg; mannitol: 210 mg; pH: 6.8

| | | | | | | |
|---|---|---|---|---|---|---|
| Initial | NA | 4.49 | 0.88 | 0.74 | 7.39 | — |
| 1 M* | NA | 6.29 | 1.13 | 0.71 | 9.54 | 2.15 |

Batch B; Supplier #2; Daptomycin: 350 mg; L-arginine HCl: 42 mg; mannitol: 210 mg; pH: 6.8

| | | | | | | |
|---|---|---|---|---|---|---|
| Initial | NA | 4.52 | 0.35 | 0.37 | 6.86 | — |
| 1 M* | NA | 7.48 | 0.42 | 0.30 | 9.57 | 2.71 |

*= Stability study terminated after this time point
^{#}HPLC Method-1 was subsequently determined to result in the co-elution of the lactone hydrolysis impurity (Impurity A) and Impurity-1, therefore the sum of these two impurities is reported as the “lactone impurity”; see Example 5 for further details.

### Example 3: Daptomycin Compositions Containing L-Arginine HCl Salt

Each of the formulations in Table 7 contained daptomycin (350 mg/vial) in water or water/tertiary-butyl alcohol (TBA, in a concentration of 0.1 mL TBA per mL water), and the indicated amount (mg/vial) of L-arginine hydrochloride salt. The pH of the solution was adjusted using NaOH and/or HCl or phosphoric acid and/or NaOH. The solvent was evaporated during lyophilization. The general manufacturing process disclosed in Example 1 was used to manufacture the below compositions and the compositions were analyzed using the HPLC Method-1.

TABLE 7
Daptomycin Compositions 350 mg/vial Containing L-Arginine HCl

| | Arginine | | | pH | |
|---|---|---|---|---|---|
| | HCl | Molar | | adjusting | |
| Batch No | (mg/vial) | Ratio* | pH | Agent | Solvent |
| C | 28 | 1:0.6 | 6.8 | 5N NaOH | WFI |
| D | 56 | 1:1.2 | 6.8 | 5N NaOH | WFI |
| (Supplier #1) | | | | | |
| E | 196 | 1:4.3 | 6.8 | NaOH | WFI & t-butyl |
| (Supplier #2) | | | | | alcohol (TBA) |
| | | | | | (0.1 ml/ml) |
| F | 210 | 1:4.6 | 6.8 | 5N NaOH | WFI |
| (Supplier #2) | | | | | |
| G | 210 | 1:4.6 | 6.8 | 5N NaOH | WFI |
| (Supplier #1) | | | | | |
| H | 210 | 1:4.6 | 6.8 | 2N NaOH | WFI |
| (Supplier #2) | | | | | |
| I | 525 | 1:11.5 | 6.8 | 2N NaOH | WFI |
| (Supplier #2) | | | | | |
| J | 525 | 1:11.5 | 6.8 | 1N Phosphoric | WFI |
| (Supplier #1) | | | | acid & 2N | |
| | | | | NaOH | |

*Molar ratio is calculated considering the molecular weight of Daptomycin: 1620.67 and L-arginine HCl: 210.66.

The formulations were analyzed for stability under various conditions, including accelerated stability conditions (40° C./75% o RH). Stability studies were discontinued at the 1-month, or 2-month time point if the formulations failed to exhibit acceptable stability. However, for formulations showing promising initial stability data, the studies were extended to longer durations. As noted earlier, HPLC Method-1 resulted in the co-elution of the lactone hydrolysis impurity (Impurity A) and Impurity 1; hence both these impurities are reported as the sum of “lactone hydrolysis and Impurity 1” in Table 7(a). For some of the later stability time points for these formulations, HPLC Method-2 (in Example 5), which separates the two impurities, was employed. In such cases, both the combined total of these impurities and their individual levels are reported.

TABLE 7(a)
Stability Data for Daptomycin (350 mg/vial) Compositions Containing L-Arginine Hydrochloride

| | | | | | | | | |
|---|---|---|---|---|---|---|---|---|
| | | Sum of Lactone | Lactone | | Anhydro | Beta- | Total | Δ (Total |
| Time Point | Assay | Hydrolysis and | Hydrolysis | Impurity-1 | daptomycin | Isomer | Impurities | Impurities) |
| (Month, M) | (%) | Impurity 1 (%) | (%) | (%) | (%) | (%) | (%) | % |

Batch C; Supplier #1; Daptomycin: 350 mg; L-arginine HCl: 28 mg; pH: 6.8
a) 40° C./75% RH

| Initial | | 104.4 | 1.32 | NA | NA | 0.46 | 0.24 | 3.24 | — |
|---|---|---|---|---|---|---|---|---|---|
| 1 | M | 104.1 | 2.92 | NA | NA | 0.81 | 0.33 | 5.95 | 2.71 |
| 3 | M* | 98.4 | 3.96 | NA | NA | 1.03 | 0.22 | 6.79 | 3.55 |

b) 25° C./60% RH

| | | | | | | | | | |
|---|---|---|---|---|---|---|---|---|---|
| 3 | M* | 101.3 | 2.04 | NA | NA | 0.7 | 0.21 | 4.3 | 1.06 |

Batch D; Supplier #1; Daptomycin: 350 mg; L-arginine HCl: 56 mg; pH: 6.8
a) 40° C./75% RH

| Initial | | NA | 1.99 | NA | NA | 1.01 | 0.64 | 4.9 | — |
|---|---|---|---|---|---|---|---|---|---|
| 1 | M* | NA | 4.01 | NA | NA | 1.18 | 0.60 | 7.01 | 2.11 |

Batch E; Supplier #2; Daptomycin: 350 mg; L-arginine HCl: 196 mg; pH: 6.8
a) 40° C./75% RH

| Initial | | 101.2 | 0.62 | NA | NA | 1.1 | 0.77 | 3.75 | — |
|---|---|---|---|---|---|---|---|---|---|
| 1 | M | 99.6 | 1.50 | NA | NA | 1.2 | 0.56 | 4.85 | 1.1 |
| 3 | M | 100.9 | 2.33 | NA | NA | 1.42 | 0.67 | 6.00 | 2.25 |
| 6 | M | 98.1 | 2.72 | NA | NA | 1.42 | 0.58 | 6.22 | 2.47 |

b) 25° C./60% RH

| | | | | | | | | | |
|---|---|---|---|---|---|---|---|---|---|
| 6 | M | 100.4 | 1.13 | NA | NA | 1.12 | 0.54 | 4.23 | 0.48 |
| 18 | M | 99.2 | 1.75 | 0.52 | 1.23 | 1.19 | 0.49 | 5.12 | 1.37 |

Batch F; Supplier #2; Daptomycin: 350 mg; L-arginine HCl: 210 mg; pH: 6.8
a) 40° C./75% RH

| Initial | | 97.9 | 0.7 | NA | NA | 1.3 | 0.5 | 3.7 | — |
|---|---|---|---|---|---|---|---|---|---|
| 1 | M | 96.2 | 1.69 | NA | NA | 1.4 | 0.39 | 5.12 | 1.42 |
| 3 | M | 98.0 | 2.18 | NA | NA | 1.53 | 0.37 | 5.54 | 1.84 |
| 6 | M | 96.9 | 2.63 | NA | NA | 1.54 | 0.56 | 5.93 | 2.23 |

a) 25° C./60% RH

| | | | | | | | | | |
|---|---|---|---|---|---|---|---|---|---|
| 6 | M | 97.6 | 1.16 | NA | NA | 1.30 | 0.34 | 4.04 | 0.34 |
| 18 | M | 97 | 1.7 | 0.48 | 1.22 | 1.37 | 0.36 | 5.05 | 1.35 |

Batch G; Supplier #1; Daptomycin: 350 mg; L-arginine HCl: 210 mg; pH: 6.8
a) 40° C./75% RH

| Initial | | 99.6 | 0.28 | NA | bb | 0.66 | 0.15 | 2.35 | — |
|---|---|---|---|---|---|---|---|---|---|
| 1 | M | 99.4 | 1.18 | NA | NA | 0.81 | 0.1 | 3.56 | 1.21 |
| 3 | M | 100.0 | 2.1 | NA | NA | 0.96 | 0.12 | 4.74 | 2.39 |
| 6 | M | 99.5 | 2.66 | NA | NA | 0.96 | 0.1 | 5.14 | 2.79 |

b) 25° C./60% RH

| | | | | | | | | | |
|---|---|---|---|---|---|---|---|---|---|
| 6 | M | 100.5 | 0.84 | NA | NA | 0.66 | 0.08 | 2.95 | 0.6 |
| 18 | M | 97.4 | 1.47 | 0.21 | 1.26 | 0.79 | 0.08 | 4.03 | 1.68 |

Batch H; Supplier #2; Daptomycin: 350 mg; L-arginine HCl: 210 mg; pH: 6.8
a) 40° C./75% RH

| Initial | | 102.7 | 0.48 | NA | NA | 1.08 | 0.31 | 3.32 | — |
|---|---|---|---|---|---|---|---|---|---|
| 1 | M | 98.6 | 1.32 | NA | NA | 1.19 | 0.30 | 4.41 | 1.09 |
| 3 | M | 97.4 | 2.10 | NA | NA | 1.32 | 0.37 | 5.34 | 2.02 |
| 6 | M | 98.9 | 2.66 | NA | NA | 1.53 | 0.39 | 6.64 | 3.32 |

b) 25° C./60% RH

| | | | | | | | | | |
|---|---|---|---|---|---|---|---|---|---|
| 6 | M | 99.5 | 1.15 | NA | NA | 1.2 | 0.38 | 4.29 | 0.97 |
| 18 | M | 100.6 | 2.4 | 0.43 | 1.97 | 1.25 | 0.34 | 5.6 | 2.28 |
| 24 | M | 102.5 | 2.36 | 0.42 | 1.94 | 1.25 | 0.28 | 5.39 | 2.07 |

Batch I; Supplier #2; Daptomycin: 350 mg; L-arginine HCl: 525 mg; pH: 6.8
40° C./75% RH

| Initial | | 103 | 0.2 | NA | NA | 0.91 | 0.14 | 2.70 | — |
|---|---|---|---|---|---|---|---|---|---|
| 1 | M | 98.9 | 2.15 | NA | NA | 1.11 | 0.16 | 4.74 | 2.04 |
| 2 | M* | 89.5 | 7.96 | NA | NA | 1.81 | 0.37 | 11.67 | 8.97 |

Batch J; Supplier #1; Dap: 350 mg; L-arginine HCl: 525 mg; pH: 6.8
40° C./75% RH

| Initial | | 100.6 | 0.23 | NA | NA | 0.71 | 0.08 | 2.44 | — |
|---|---|---|---|---|---|---|---|---|---|
| 1 | M | 96.8 | 3.17 | NA | NA | 1.00 | 0.13 | 5.67 | 3.23 |
| 2 | M* | 89.7 | 7.92 | NA | NA | 1.43 | 0.17 | 11.11 | 8.67 |

NA = Not applicable. As these samples were analyzed using the HPLC Method-1 which co-eluted lactone hydrolysis impurity (Impurity A) and Impurity 1.
*Stability study terminated after this point.

The increase in total impurities under accelerated conditions (40° C./75% RH) at 6 months is less for a pharmaceutical composition containing 350 mg of daptomycin and 210 mg of L-arginine HCl when compared to other weight ratios of daptomycin to L-arginine HCl. The inventors surprisingly found that a molar ratio of daptomycin to L-arginine HCl in the range of about 1:3.5 to about 1:7, e.g., about 1:6; or about 1:4.6, provides a more stable daptomycin composition when compared to other molar ratios, as shown in Table 8.

The inventors tested the formulations containing daptomycin and L-arginine HCl in a molar ratio of about 1:11.5, which resulted in higher level of impurities at accelerated conditions (40° C./75% RH) for 2 months. Therefore, the stability study was terminated at this time point.

As provided in Example 1, Table 4, the daptomycin formulation containing sucrose stored at 25° C./60% RH for 18 months exhibits about a 1.4% increase in total impurities from the initial level. The daptomycin formulations containing L-arginine HCl in a molar ratio of about 1:4.6 exhibit a similar trend (about 1.5% difference in total impurities when compared to the initial level at 25° C./60% RH for 18 months).

### Example 3b: Daptomycin Composition Containing L-Arginine Free Base

The general manufacturing process disclosed in Example 1 was used to manufacture the compositions shown in Table 7(b), and the compositions were analyzed using HPLC Method-1.

TABLE 7(b)
Daptomycin Composition Containing
L-Arginine Base as a Stabilizer

| | | | | pH | |
|---|---|---|---|---|---|
| | L-Arginine | Molar | | Adjusting | |
| Batch No. | (mg/vial) | Ratio* | pH | Agent | Solvent |
| Batch K | 187 | 1:5 | 6.8 | 1N Phosphoric | WFI & TBA |
| (Supplier #2) | | | | acid | (0.1 ml/ml) |
| Batch L | 187 | 1:5 | 6.8 | 2N Phosphoric | WFI |
| (Supplier #2) | | | | acid & 2N | |
| | | | | NaOH | |
| Batch M | 187 | 1:5 | 6.8 | 2N Phosphoric | WFI |
| (Supplier #1) | | | | acid & 2N | |
| | | | | NaOH | |

*Molar ratio is calculated considering the molecular weight of daptomycin: 1620.67 & L-arginine: 174.2

The daptomycin (350 mg) formulations containing L-arginine free base (187 mg) exhibit about a 3.5% (by HPLC) increase in total impurities from an initial time point to 6 months at 40° C./75% RH, whereas the formulations containing L-arginine HCl salt (196 mg) results in less than about 2.75% (by HPLC) increase in total impurities in the same conditions. It should be noted that L-arginine phosphate salt (formed from the phosphoric acid used for pH adjustment) containing formulations exhibit a high level of increase in total impurities from an initial time point to 6 months at accelerated conditions (40° C./75% RH) when compared to L-arginine HCl salt containing compositions at the same conditions.

TABLE 7(c)
Stability Data of Daptomycin Compositions Containing Arginine Base

| | | Sum of | | | | | | |
|---|---|---|---|---|---|---|---|---|
| | | Lactone | | | | | | Δ |
| | | Hydrolysis + | Lactone | | Anhydro | Beta- | Total | (total |
| Time Point | Assay | Impurity-1 | hydrolysis | Impurity-1 | daptomycin | Isomer | Impurities | impurities) |
| (Month, M) | (%) | (%) | (%) | (%) | (%) | (%) | (%) | (%) |

Batch K; Supplier #2; Daptomycin: 350 mg; L-arginine: 187 mg; pH: 6.8
a) 40° C./75% RH

| Initial | | 101.9 | 0.47 | NA | NA | 0.77 | 0.79 | 3.92 | — |
|---|---|---|---|---|---|---|---|---|---|
| 1 | M | 99.9 | 1.71 | NA | NA | 1.26 | 1.01 | 5.22 | 1.3 |
| 3 | M | 96.6 | 2.42 | NA | NA | 1.14 | 0.77 | 6.21 | 2.29 |
| 6 | M | 97.8 | 3.17 | NA | NA | 1.35′ | 0.9 | 7.49 | 3.57 |

b) 25° C./60% RH

| | | | | | | | | | |
|---|---|---|---|---|---|---|---|---|---|
| 6 | M | 99 | 1.47 | NA | NA | 0.97 | 0.82 | 5.02 | 1.10 |
| 15 | M | 99.9 | 2.67 | 0.99 | 1.68 | 0.99 | 0.66 | 5.24 | 1.32 |

Batch L; Supplier #2; Daptomycin: 350 mg; L-arginine: 187 mg; pH: 6.8
a) 40° C./75% RH

| Initial | | 103.1 | 0.29 | NA | NA | 1.19 | 0.19 | 2.95 | — |
|---|---|---|---|---|---|---|---|---|---|
| 1 | M | 100.3 | 1.55 | NA | NA | 1.38 | 0.22 | 4.80 | 1.85 |
| 3 | M | 97.5 | 2.38 | NA | NA | 1.56 | 0.29 | 5.93 | 2.98 |
| 6 | M | 98.5 | 2.82 | NA | NA | 1.5 | 0.33 | 6.5 | 3.55 |

b) 25° C./60% RH

| | | | | | | | | | |
|---|---|---|---|---|---|---|---|---|---|
| 6 | M | 101.4 | 1.42 | NA | NA | 1.48 | 0.24 | 4.98 | 2.03 |
| 15 | M | 100.3 | 1.79 | 0.25 | 1.54 | 1.25 | 0.27 | 5.35 | 2.40 |
| 18 | M | 102.3 | 1.92 | 0.26 | 1.66 | 1.24 | 0.34 | 5.2 | 2.25 |

Batch M; Supplier #1; Daptomycin: 350 mg; L-arginine: 187 mg; pH: 6.8
a) 40° C./75% RH

| Initial | | 101.6 | 0.35 | NA | NA | 0.86 | 0.08 | 3.2 | — |
|---|---|---|---|---|---|---|---|---|---|
| 1 | M | 101.6 | 1.38 | NA | NA | 0.92 | 0.11 | 3.91 | 0.71 |
| 3 | M | 96.5 | 2.07 | NA | NA | 1.10 | 0.14 | 5.59 | 2.39 |
| 6 | M | 97.7 | 2.47 | NA | NA | 1.41 | 0.19 | 6.48 | 3.28 |

b) 25° C./60% RH

| | | | | | | | | | |
|---|---|---|---|---|---|---|---|---|---|
| 6 | M | 98.1 | 1.3 | NA | NA | 1.3 | 0.11 | 4.08 | 2.49 |
| 18 | M | 98.3 | 2.35 | 0.32 | 2.03 | 0.98 | 0.29 | 6 | 3.8 |
| 24 | M | 98.9 | 2.33 | 0.29 | 2.04 | 0.99 | 0.22 | 6 | 3.8 |

### Example 4: Arginine Concentration

Daptomycin (500 mg/vial) compositions having different concentrations (240 mg, 300 mg and 360 mg) of L-arginine HCl were prepared according to the general manufacturing process and their stability determined under accelerated conditions (40° C./75% RH). The results are provided in Table 8.

As can be seen from Table 8, the daptomycin composition containing 300 mg and 360 mg of L-arginine HCl exhibits no more than about 1% (by HPLC) increase in total impurities upon storage for 6 months at 40° C. and 75% relative humidity. However, the daptomycin composition containing 300 mg L-arginine HCl surprisingly exhibits a lower level of total impurities at initial and 6M time points when compared to the daptomycin composition containing 360 mg L-arginine HCl.

The composition containing 240 mg L-arginine HCl results in more than about 1% (by HPLC) increase of total impurities under the same conditions.

TABLE 8
Stability Comparison of Daptomycin (500 mg/vial) Compositions with Different
L-Arginine HCl Concentrations under Accelerated Conditions (40° C./75% RH)

| | | | | | | | |
|---|---|---|---|---|---|---|---|
| | | Lactone | | Anhydro | Beta- | Total | Δ (Total |
| Time Point | Assay | Hydrolysis | Impurity-1 | daptomycin | Isomer | Impurities | Impurities) |
| (Month, M) | (%) | (%) | (%) | (%) | (%) | (%) | (%) |

a) 240 mg L-Arginine HCl, pH 5.25

| | | | | | | | |
|---|---|---|---|---|---|---|---|
| Initial | 99 | 0.12 | 0.09 | 1.33 | 0.16 | 3.47 | — |
| 6 M | 99.7 | 0.13 | 0.80 | 2.08 | 0.17 | 5.12 | 1.65 |

b) 300 mg L-Arginine HCl, pH 5.25

| | | | | | | | |
|---|---|---|---|---|---|---|---|
| Initial | 104 | 0.08 | 0.08 | 0.89 | 0.12 | 2.81 | — |
| 6 M | 104.2 | 0.09 | 0.65 | 1.51 | 0.14 | 3.96 | 1.15 |

c) 360 mg L-Arginine HCl, pH 5.25

| | | | | | | | |
|---|---|---|---|---|---|---|---|
| Initial | 100 | 0.16 | 0.08 | 1.30 | 0.15 | 3.48 | — |
| 6 M | 98.4 | 0.18 | 0.59 | 1.67 | 0.15 | 4.48 | 1.0 |

### Example 5: HPLC Method-2 for Relative Substances of Daptomycin for Injection

Although the daptomycin compositions using L-arginine (HCl or phosphate) showed acceptable stability in terms of total impurities, the most significant degradation impurity formed during accelerated stability is the putative “lactone hydrolysis” impurity. This is surprising because it has been established that the formation of the lactone hydrolysis impurity is favored under alkaline conditions, whereas these compositions are targeted to neutral pH (6.0 to 7.0).

Further to understand this anomaly, an alternate HPLC method (HPLC Method-2) was developed with a longer run time and longer retention time for the main daptomycin peak (˜65 minutes) compared to the HPLC method in U.S. Pat. No. 8,835,832 (HPLC Method-1, daptomycin retention of about 36.0±1.5 minutes).

| Method | |
|---|---|
| Parameter/Attribute | Condition/ Criteria |
| Mobile Phase | pH 3.6 potassium dihydrogen phosphate buffer |
| | (aqueous) and acetonitrile (670 ± 1 g to 255 ± 1 g) |
| Blank/Diluent | Water and acetonitrile (90:10% v/v) |
| Column | YMC Triart, C18, 250 mm × 4.6 mm, 3 μm |
| | (Part No.: TA12S03-2546WT) or equivalent |
| Flow rate | 0.8 mL/min |
| Injection volume | 30 μL |
| Detector | 223 nm |
| Run time | 110 minutes |
| Column temperature | 35° C. |
| Sample temperature | 5° C. |
| Retention Time | Approximately 65 minutes for daptomycin peak |

It was discovered that the prior art method (HPLC Method-1) disclosed in Table 3 of U.S. Pat. No. 8,835,382 results in the co-elution of two different impurities: the lactone hydrolysis product and Impurity 1. The HPLC method described herein (Method-2) can resolve the co-elution of these two impurities allowing determination of each impurity separately instead of as a sum. The use of HPLC Method-2 has led to the surprising discovery that Impurity 1, not previously described as a significant degradation product in daptomycin formulations, is an important degradation product to control in daptomycin formulations. Due to separation of these two impurities, the present inventors were able to minimize the levels of these individual impurities and develop daptomycin formulations that exhibit enhanced stability.

### Example 6: Stability Profile of Daptomycin Compositions at Different pH

A lyophilized daptomycin composition is obtained from a compounded solution containing daptomycin and L-arginine or its HCl salt wherein the pH of the composition is about 4.5 to about 7. The compositions containing daptomycin and L-arginine HCl salt in the molar ratio of about 1:3.8 to 1:5.5 and a pH of from about 4.5 to about 7 exhibit less than about 4% (by HPLC), e.g., less than about 2.75% (by HPLC), increase in total impurities from an initial time point to 6 months at 40° C. and 75% RH.

The daptomycin compositions containing L-arginine HCl and having a pH of from about 5 to about 6, e.g., about 5.5 exhibit less than about 2.25% (by HPLC) increase in total impurities from an initial time point to 6 months at 40° C. and 75% RH. The pH may be adjusted using NaOH, HCl, phosphoric acid, and the like, and any combination thereof. In one embodiment, the pH is adjusted using hydrochloric acid.

Further, the present invention also surprisingly provides daptomycin compositions comprising daptomycin and L-arginine HCl salt, where the pH of the composition is about 5 to about 6, and where the composition exhibits no more than about 1.5% (by HPLC) increase of total impurities from an initial time point to 3 months at 40° C. and 75% RH.

In an embodiment, the present invention provides a pharmaceutical composition comprising daptomycin and L-arginine HCl salt having a pH of from about 4.5 to about 6, e.g., from about 5.25 to about 5.5, wherein the composition is reconstituted within about 10 minutes, e.g., within about 8 minutes, or within about 5 minutes.

Daptomycin formulation batches N-T, and V were manufactured using the common manufacturing process provided in Example 1. These batches were analyzed using HPLC Method-2.

TABLE 9 (a)
Stability Data of Daptomycin (500 mg) Compositions Containing L-Arginine HCl
(300 mg) at Different pH Values under Accelerated Conditions (40° C./75% RH)

| | | | | | | | | Δ | |
|---|---|---|---|---|---|---|---|---|---|
| | | | | Lactone | Beta- | Anhydro | Total | (Total | Recon. |
| Batch | Target | Time Point | Imp-1 | hydrolysis | Isomer | daptomycin | Impurities | Impurities) | time |
| # | pH | (Month, M) | (%) | (%) | (%) | (%) | (%) | (%) | (min:sec) |
| N | 4.50 | Initial | 0.03 | 0.1 | 0.13 | 1.03 | 3.1 | — | 3:10 |
| N | 4.50 | 1 M | 0.1 | 0.1 | 0.14 | 1.27 | 3.46 | 0.36 | 5:20 |
| N | 4.50 | 3 M | 0.17 | 0.1 | 0.15 | 1.35 | 3.63 | 0.53 | 4:20 |
| N | 4.50 | 6 M | 0.24 | 0.11 | 0.19 | 1.61 | 4.06 | 0.96 | 5:30 |
| O | 5.00 | Initial | 0.036 | 0.16 | 0.15 | 1.04 | 3.20 | — | 3:55 |
| O | 5.00 | 1 M | 0.16 | 0.16 | 0.15 | 1.26 | 3.58 | 0.38 | 6:35 |
| O | 5.00 | 3 M | 0.28 | 0.16 | 0.16 | 1.35 | 3.77 | 0.57 | 5:20 |
| O | 5.00 | 6 M | 0.42 | 0.17 | 0.18 | 1.61 | 4.25 | 1.05 | 5:00 |
| P | 5.25 | Initial | 0.08 | 0.21 | 0.1 | 0.95 | 3.03 | — | 4:15 |
| P | 5.25 | 1 M | 0.34 | 0.2 | 0.13 | 1.07 | 3.41 | 0.38 | 4:05 |
| P | 5.25 | 3 M | 0.53 | 0.22 | 0.15 | 1.26 | 3.88 | 0.85 | 4:23 |
| P | 5.25 | 6 M | 0.71 | 0.22 | 0.15 | 1.34 | 4.12 | 1.09 | 4:28 |
| Q | 5.50 | Initial | NA | 0.23 | 0.14 | 0.9 | 2.98 | — | 4:10 |
| Q | 5.50 | 1 M | 0.39 | 0.18 | 0.15 | 0.86 | 3.06 | 0.08 | 5:50 |
| O | 5.50 | 3 M | 0.7 | 0.18 | 0.17 | 0.98 | 3.65 | 0.67 | 6:10 |
| Q | 5.50 | 6 M | 1.07 | 0.2 | 0.21 | 1.2 | 4.46 | 1.48 | 4:50 |
| R | 6.00 | Initial | NA | 0.17 | 0.09 | 0.77 | 2.61 | — | 2:52 |
| R | 6.00 | 1 M | NA | 0.81 | 0.15 | 0.88 | 3.43 | 0.82 | 3:50 |
| R | 6.00 | 3 M | 1.01 | 0.14 | 0.15 | 1.03 | 4.07 | 1.46 | 4:10 |
| R | 6.00 | 6 M | 1.47 | 0.16 | 0.14 | 1.15 | 5.21 | 2.6 | 4:15 |
| S | 6.50 | Initial | NA | 0.24 | 0.08 | 0.74 | 2.6 | — | 3:45 |
| S | 6.50 | 1 M | NA | 1.04 | 0.13 | 0.94 | 3.10 | 0.5 | 3:10 |
| S | 6.50 | 3 M | 1.27 | 0.2 | 0.16 | 0.94 | 4.33 | 1.73 | 2:59 |
| S | 6.50 | 6 M | 1.85 | 0.22 | 0.16 | 1.05 | 4.74 | 2.14 | 3:52 |
| T | 7.00 | Initial | NA | 0.17 | 0.08 | 0.75 | 2.69 | — | 6:92 |
| T | 7.00 | 1 M | NA | 1.07 | 0.15 | 1.00 | 3.92 | 1.23 | 6:90 |
| T | 7.00 | 3 M | 1.35 | 0.16 | 0.17 | 0.91 | 4.33 | 1.64 | 4:10 |
| T | 7.00 | 6 M | 1.97 | 0.15 | 0.18 | 1.02 | 4.80 | 2.11 | 4:05 |

Recon. Time = Reconstitution Time

Daptomycin formulation batches U-Z were manufactured using the common manufacturing process provided in Example 1. These batches were analyzed using HPLC Method-2.

TABLE 9(b)
Stability Data of Daptomycin (350 mg) Compositions Containing L-Arginine HCl
(210 mg) at Different pH Values under Accelerated Conditions (40° C./75% RH)

| | | | | | | | | Δ | |
|---|---|---|---|---|---|---|---|---|---|
| | | | | Lactone | Beta- | Anhydro | Total | (Total | Recon. |
| Batch | Target | Time Point | Impurity-1 | hydrolysis | Isomer | daptomycin | Impurities | Impurities) | Time |
| # | pH | (Month, M) | (%) | (%) | (%) | (%) | (%) | (%) | (Min:Sec) |
| U | 5.25 | Initial | 0.1 | 0.08 | 0.13 | 0.88 | 2.87 | — | 4:05 |
| U | 5.25 | 1 M | 0.34 | 0.09 | 0.15 | 1.19 | 3.47 | 0.60 | 3:50 |
| U | 5.25 | 3 M | 0.54 | 0.07 | 0.13 | 1.2 | 3.63 | 0.76 | 3:50 |
| U | 5.25 | 6 M | 0.7 | 0.09 | 0.11 | 1.42 | 3.96 | 1.09 | 4:35 |
| V | 5.5 | Initial | NA | 0.11 | 0.14 | 0.75 | 2.65 | — | 1:45 |
| V | 5.5 | 1 M | 0.38 | 0.09 | 0.15 | 0.85 | 3.06 | 0.41 | 3:10 |
| V | 5.5 | 3 M | 0.74 | 0.09 | 0.12 | 1.12 | 3.36 | 0.71 | 4:48 |
| V | 5.5 | 6 M | 1.83 | 0.14 | 0.22 | 1.04 | 4.98 | 2.33 | 3:58 |
| W | 6.0 | Initial | NA | 0.17 | 0.09 | 0.76 | 2.6 | — | 2:40 |
| W | 6.0 | 1 M | NA | 0.63 | 0.14 | 0.83 | 3.15 | 0.55 | 2:45 |
| W | 6.0 | 3 M | 0.96 | 0.14 | 0.16 | 0.9 | 4.02 | 1.21 | 2:50 |
| W | 6.0 | 6 M | 1.47 | 0.15 | 0.15 | 1.17 | 4.5 | 1.69 | 4:05 |
| X | 6.25 | Initial | NA | 0.15 | 0.18 | 0.69 | 2.66 | — | 4:20 |
| X | 6.25 | 1 M | 0.71 | 0.11 | 0.15 | 0.78 | 3.27 | 0.61 | 4:30 |
| X | 6.25 | 3 M | 1.3 | 0.12 | 0.12 | 0.97 | 3.8 | 1.14 | 3:10 |
| X | 6.25 | 6 M | 1.83 | 0.14 | 0.23 | 1.02 | 4.96 | 2.3 | 3:25 |
| Y | 6.5 | Initial | NA | 0.24 | 0.11 | 0.74 | 2.66 | — | 3:10 |
| Y | 6.5 | 1 M | NA | 1.08 | 0.14 | 0.84 | 3.64 | 0.98 | 3:20 |
| Y | 6.5 | 3 M | 1.32 | 0.21 | 0.18 | 0.96 | 4.4 | 1.51 | 2:59 |
| Y | 6.5 | 6 M | 1.96 | 0.28 | 0.17 | 1.06 | 4.96 | 2.04 | 4:15 |
| Z | 7 | Initial | NA | 0.17 | 0.12 | 0.75 | 2.74 | — | 2:35 |
| Z | 7 | 1 M | NA | 1.16 | 0.18 | 0.86 | 3.94 | 1.2 | 3:45 |
| Z | 7 | 3 M | 1.43 | 0.13 | 0.19 | 0.9 | 4.37 | 1.63 | 3:30 |
| Z | 7 | 6 M | 2.10 | 0.15 | 0.19 | 1.02 | 5.01 | 2.27 | 3:25 |

Recon. Time = Reconstitution Time

HPLC Method-1 elutes the “lactone hydrolysis” impurity (Impurity A) at about 0.72 RRT (relative retention time) without any peak separation. During the development of the daptomycin for injection formulations described herein, using HPLC Method-2, the inventors surprisingly found that the peak corresponding to the lactone hydrolysis impurity obtained using HPLC Method-1 co-eluted the lactone hydrolysis impurity and Impurity-1 together. In contrast, HPLC Method-2 can detect, separate and elute the lactone hydrolysis impurity and Impurity-1 separately, at about 0.71 RRT and 0.75 RRT respectively.

The pharmaceutical compositions of daptomycin having a pH of from about 5 to about 6, e.g., from about 5.25 to about 5.5, exhibit enhanced stability compared to the compositions having a pH above 6. The daptomycin compositions described herein exhibit, in one embodiment, no more than about 0.6% (by HPLC) increase in Impurity-1 upon storage for 3 months at 40° C. and 75% relative humidity.

As provided in Tables 9(a) and (b), daptomycin compositions containing L-arginine HCl having a pH from about 5.0 to about 5.5 exhibit, in one embodiment, no more than about 0.75% (by HPLC) increase in Impurity-1 and no more than about 1% (by HPLC) increase in total impurities upon storage for 3 months at 40° C. and 75% relative humidity. A daptomycin composition having a pH of about 6 exhibits about 1.5% (by HPLC) increase in total impurities upon storage for 3 months at 40° C. and 75% relative humidity and exhibits an increase in Impurity-1 (greater than about 0.6%) under the same conditions when compared to a composition having a pH between 5 and 6.

In addition to providing a stable daptomycin composition, the present invention also provides daptomycin compositions which may be rapidly reconstituted (dissolves) in a pharmaceutically acceptable diluent. For example, compositions containing daptomycin and L-arginine HCl in a molar ratio of about 1:4.6 and having a pH of about 5.25 may be reconstituted in WFI or 0.9% NaCl in less than about 10 minutes; e.g., in about 4-6 minutes or less. As shown in Table 10, the reconstitution time for CUBICIN® is about 13-17 minutes. The reconstitution time of a daptomycin composition according to the present invention is faster than CUBICIN®.

TABLE 10
Comparison of Reconstitution Time

| | | Reconstitution |
|---|---|---|
| Batch # | pH | Time (min:sec) |
| Cubicin | 4.66 | 13:56 to 17:10 |
| (Multiple RLD lots) | | |

A daptomycin composition containing L-arginine HCl (molar ratio of about 1:4.6) having a pH greater than about 6 was reconstituted in a diluent in less than about 5 minutes. However, as mentioned above, the higher pH (greater than about 6) results in a higher level of Impurity-1 and total impurities when compared to a similar composition at a pH of about 5.25.

The present inventors have surprisingly arrived, in one embodiment, at a composition comprising daptomycin and L-arginine HCl in a molar ratio of about 1:4.6 and having a pH of about 5.25 that exhibits a low amount of Impurity-1 and total impurities, and may be reconstituted rapidly (e.g., within about 5 minutes or less).

TABLE 11(a)
Stability Data of Daptomycin (500 mg) Compositions Containing L-Arginine HCl (300 mg) at Different
pH Values under Long-term Conditions (25° C./60% RH) along with Reconstitution Time

| | | | | | | | | Δ | |
|---|---|---|---|---|---|---|---|---|---|
| | | | | Lactone | Beta- | Anhydro | Total | (Total | Recon. |
| Batch | | Time Point | Imp-1 | Hydrolysis | Isomer | daptomycin | Impurities | Impurities) | Time |
| # | pH | (Month, M) | (%) | (%) | (%) | (%) | (%) | (%) | (min:sec) |
| N | 4.50 | Initial | 0.03 | 0.1 | 0.13 | 1.03 | 3.1 | — | 03:10 |
| N | 4.50 | 6 M | 0.11 | 0.11 | 0.14 | 1.25 | 3.46 | 0.36 | 05:20 |
| N | 4.50 | 12 M | 0.13 | 0.1 | 0.13 | 1.33 | 3.62 | 0.52 | 04:32 |
| N | 4.50 | 18 M | 0.13 | 0.011 | 0.16 | 1.29 | 3.61 | 0.51 | NA |
| O | 5.00 | Initial | 0.036 | 0.16 | 0.15 | 1.04 | 3.2 | — | 03:55 |
| O | 5.00 | 6 M | 0.15 | 0.18 | 0.15 | 1.22 | 3.47 | 0.27 | 04:50 |
| O | 5.00 | 12 M | 0.18 | 0.17 | 0.18 | 1.29 | 3.59 | 0.39 | 04:01 |
| O | 5.00 | 18 M | 0.21 | 0.16 | 0.17 | 1.26 | 3.62 | 0.42 | NA |
| P | 5.25 | Initial | 0.08 | 0.21 | 0.1 | 0.95 | 3.03 | — | 04:15 |
| P | 5.25 | 6 M | 0.28 | 0.21 | 0.13 | 1.07 | 3.3 | 0.27 | 04:16 |
| P | 5.25 | 12 M | 0.37 | 0.22 | 0.11 | 1.14 | 3.39 | 0.36 | 04:48 |
| Q | 5.50 | Initial | NA | 0.23 | 0.14 | 0.9 | 2.98 | — | 04:10 |
| Q | 5.50 | 6 M | 0.38 | 0.19 | 0.19 | 0.92 | 3.37 | 0.39 | 04:20 |
| Q | 5.50 | 12 M | 0.49 | 0.19 | 0.14 | 0.98 | 3.56 | 0.58 | 04:23 |
| Q | 5.50 | 18 M | 0.58 | 0.18 | 0.18 | 0.91 | 3.5 | 0.52 | 04:16 |
| R | 6.00 | Initial | NA | 0.17 | 0.09 | 0.77 | 2.61 | — | 2:52 |
| R | 6.00 | 6 M | 0.49 | 0.14 | 0.13 | 0.91 | 3.7 | 1.09 | 3:55 |
| R | 6.00 | 12 M | 0.74 | 0.13 | 0.14 | 0.94 | 3.54 | 0.93 | 4:05 |
| R | 6.00 | 18 M | 0.83 | 0.13 | 0.17 | 0.94 | 3.73 | 1.12 | NA |
| S | 6.50 | Initial | NA | 0.24 | 0.08 | 0.74 | 2.6 | — | 03:45 |
| S | 6.50 | 6 M | 0.61 | 0.2 | 0.15 | 0.82 | 3.25 | 0.65 | 04:05 |
| S | 6.50 | 12 M | 0.91 | 0.18 | 0.16 | 0.88 | 3.76 | 1.16 | 04:15 |
| S | 6.50 | 18 M | 1.06 | 0.18 | 0.15 | 0.9 | 3.96 | 1.36 | NA |
| T | 7.00 | Initial | NA | 0.17 | 0.08 | 0.75 | 2.69 | — | 4:30 |
| T | 7.00 | 6 M | 0.66 | 0.12 | 0.17 | 0.80 | 3.28 | 0.59 | 04:20 |
| T | 7.00 | 12 M | 1.01 | 0.11 | 0.17 | 0.83 | 3.81 | 1.12 | 04:35 |
| T | 7.00 | 18 M | 1.13 | 0.12 | 0.18 | 0.85 | 4.03 | 1.34 | NA |

NA: Not analyzed - Recon. Time = Reconstitution Time

TABLE 11(b)
Stability Data of Daptomycin (350 mg) Compositions Containing L-Arginine HCl (210 mg) at Different
pH Values under Long-term Conditions (25° C./60% RH) along with Reconstitution Time

| | | | | | | | | Δ | |
|---|---|---|---|---|---|---|---|---|---|
| | | | | Lactone | Beta- | Anhydro | Total | (Total | Recon. |
| Batch | | Time Point | Impurity-1 | Hydrolysis | Isomer | daptomycin | Impurities | Impurities) | Time |
| # | pH | (Month, M) | (%) | (%) | (%) | (%) | (%) | (%) | (min:sec) |
| U | 5.25 | Initial | 0.1 | 0.08 | 0.13 | 0.88 | 2.87 | — | 04:05 |
| U | 5.25 | 6 M | 0.23 | 0.07 | 0.11 | 1.06 | 3.08 | 0.21 | 04:18 |
| U | 5.25 | 12 M | 0.28 | 0.07 | 0.11 | 1.03 | 3.08 | 0.21 | 04:31 |
| U | 5.25 | 18 M | 0.35 | 0.07 | 0.06 | 1.68 | 3.13 | 0.26 | 04:10 |
| V | 5.5 | Initial | NA | 0.11 | 0.14 | 0.75 | 2.65 | — | 01:45 |
| V | 5.5 | 6 M | 0.35 | 0.09 | 0.19 | 0.89 | 3.24 | 0.59 | 04:05 |
| V | 5.5 | 12 M | 0.49 | 0.09 | 0.14 | 0.95 | 3.48 | 0.83 | 03:55 |
| V | 5.5 | 18 M | 0.6 | 0.09 | 0.17 | 0.96 | 3.86 | 1.21 | 04:05 |
| W | 6.0 | Initial | NA | 0.17 | 0.09 | 0.76 | 2.6 | — | 02:40 |
| W | 6.0 | 6 M | 0.49 | 0.14 | 0.14 | 0.91 | 3.14 | 0.54 | 03:50 |
| W | 6.0 | 12 M | 0.75 | 0.12 | 0.15 | 0.89 | 3.64 | 1.04 | 03:58 |
| W | 6.0 | 18 M | 0.84 | 0.13 | 0.15 | 0.98 | 3.67 | 1.07 | NA |
| X | 6.25 | Initial | NA | 0.15 | 0.18 | 0.69 | 2.66 | — | 04:20 |
| X | 6.25 | 6 M | 0.63 | 0.12 | 0.21 | 0.78 | 2.75 | 0.09 | 03:58 |
| X | 6.25 | 12 M | 0.91 | 0.12 | 0.15 | 0.87 | 3.80 | 1.14 | 03:52 |
| X | 6.25 | 18 M | 1.05 | 0.12 | 0.18 | 0.87 | 3.86 | 1.20 | 04:03 |
| Y | 6.5 | Initial | NA | 0.24 | 0.11 | 0.74 | 2.66 | — | 3:10 |
| Y | 6.5 | 6 M | 0.62 | 0.2 | 0.16 | 0.83 | 3.28 | 0.62 | 03:55 |
| Y | 6.5 | 12 M | 0.96 | 0.19 | 0.17 | 0.83 | 3.88 | 1.22 | 04:05 |
| Y | 6.5 | 18 M | 1.1 | 0.19 | 0.18 | 0.87 | 3.95 | 1.29 | NA |
| Z | 7 | Initial | NA | 0.17 | 0.12 | 0.75 | 2.74 | — | 02:35 |
| Z | 7 | 6 M | 0.66 | 0.12 | 0.18 | 0.79 | 3.25 | 0.51 | 03:30 |
| Z | 7 | 12 M | 0.81 | 0.11 | 0.18 | 0.81 | 3.83 | 1.09 | 03:52 |
| Z | 7 | 18 M | 1.17 | 0.11 | 0.19 | 0.86 | 3.98 | 1.24 | NA |

NA: Not analyzed - Recon. Time = Reconstitution Time

### Example 7: Manufacturing Process for a Large-Scale Preparation of Daptomycin Compositions

Daptomycin compositions with batch sizes of about 25 liters to 500 liters were manufactured using the process described below.
a) approximately 20-80% by volume of the total batch volume of the solvent was added to the mixing vessel at 20-25° C.,
b) optionally, one or more stabilizers were added to the solvent at 20-25° C. and stirred until complete dissolution. Further, the pH of the solution was adjusted to the targeted value using one or more pH adjusting agent,
c) daptomycin in multiple portions (each portion containing about 1 to 10 kgs of daptomycin) was added to the solvent or excipient solution obtained in step (b) over 30-180 minutes at 2-8° C.,
d) aqueous solvent was sprinkled on any foam that was generated during and after daptomycin addition and stirred until completely dissolved,
e) optionally, the pH of the bulk solution was adjusted to the targeted pH after the addition of each portion of daptomycin,
f) optionally, a separate excipient solution containing one or more stabilizers and one or more pH adjusting agent was prepared by dissolving them in the targeted solvent,
g) the daptomycin solution obtained in step (d) was added to the excipient solution obtained in step (f),
h) optionally, the pH of the final bulk solution was adjusted to about 4.5 to about 7,
i) the batch volume was made up to the desired final volume using the solvent,
j) the bulk solution was sterile filtered using a 0.2 μm filter,
k) the sterile bulk solution was filled into vials and partially stoppered, and
l) the filled partially stoppered vials were subjected to lyophilization in a freeze dryer, followed by complete stoppering, unloading and sealing.

TABLE 12
Composition of Daptomycin Formulations

| | | | L-arginine | pH | | |
|---|---|---|---|---|---|---|
| S. | Batch | Daptomycin | HCl | Adjusting | | |
| No | Number | (mg/vial) | (mg/vial) | agent | pH | Solvent |
| 1 | AA-1 to | 500 | 300 | NaOH and/ | About | WFI* |
| | AA-3 | | | or HCl | 5.25 | |
| 2 | BB-1 to | 350 | 210 | NaOH and/ | About | WFI* |
| | BB-3 | | | or HCl | 5.25 | |

*Water for Injection - removed during lyophilization

The samples were tested for stability under accelerated (40° C. and 7500 RH) and long-term (25° C. and 60% RH) stability conditions and the stability data is provided in Tables 13-15 below.

TABLE 13
Stability Data of Daptomycin (500 mg) Containing L-Arginine
HCl (300 mg) at under Accelerated Conditions (40° C./75% RH)

| | | | | | | | | Δ |
|---|---|---|---|---|---|---|---|---|
| | | | | Lactone | Beta- | Anhydro | Total | (Total |
| Batch | | Time Point | Imp-1 | Hydrolysis | Isomer | daptomycin | Impurities | Impurities) |
| # | pH | (Month, M) | (%) | (%) | (%) | (%) | (%) | (%) |
| AA-1 | 5.25 | Initial | 0.07 | 0.11 | 0.23 | 0.92 | 3.03 | — |
| AA-1 | 5.25 | 1 M | 0.30 | 0.13 | 0.25 | 1.17 | 3.63 | 0.60 |
| AA-1 | 5.25 | 3 M | 0.50 | 0.12 | 0.25 | 1.37 | 4.02 | 0.99 |
| AA-1 | 5.25 | 6 M | 0.70 | 0.12 | 0.26 | 1.50 | 4.32 | 1.29 |
| AA-2 | 5.25 | Initial | 0.08 | 0.12 | 0.20 | 0.96 | 2.91 | — |
| AA-2 | 5.25 | 1 M | 0.34 | 0.14 | 0.14 | 1.19 | 3.35 | 0.44 |
| AA-2 | 5.25 | 3 M | 0.58 | 0.13 | 0.21 | 1.32 | 3.90 | 0.99 |
| AA-2 | 5.25 | 6 M | 0.79 | 0.14 | 0.22 | 1.49 | 4.40 | 1.49 |
| AA-3 | 5.25 | Initial | 0.07 | 0.11 | 0.18 | 0.95 | 2.85 | — |
| AA-3 | 5.25 | 1 M | 0.32 | 0.12 | 0.13 | 1.17 | 3.28 | 0.43 |
| AA-3 | 5.25 | 3 M | 0.56 | 0.12 | 0.21 | 1.30 | 3.80 | 0.99 |
| AA-3 | 5.25 | 6 M | 0.74 | 0.12 | 0.21 | 1.46 | 4.19 | 1.34 |

TABLE 14
Stability Data of Daptomycin (350 mg) Containing L-Arginine HCl (210 mg)
at Different pH Values under Accelerated Conditions (40° C./75% RH)

| | | | | | | | | Δ |
|---|---|---|---|---|---|---|---|---|
| | | | | Lactone | Beta- | Anhydro | Total | (Total |
| Batch | | Time Point | Imp-1 | Hydrolysis | Isomer | daptomycin | Impurities | Impurities) |
| # | pH | (Month, M) | (%) | (%) | (%) | (%) | (%) | (%) |
| BB-1 | 5.25 | Initial | 0.09 | 0.10 | 0.20 | 0.97 | 2.79 | — |
| BB-1 | 5.25 | 1 M | 0.36 | 0.09 | 0.19 | 1.14 | 3.29 | 0.50 |
| BB-1 | 5.25 | 3 M | 0.58 | 0.11 | 0.21 | 1.49 | 3.95 | 1.16 |
| BB-1 | 5.25 | 6 M | 0.79 | 0.10 | 0.22 | 1.42 | 4.28 | 1.49 |
| BB-2 | 5.25 | Initial | 0.07 | 0.1 | 0.19 | 0.93 | 2.86 | — |
| BB-2 | 5.25 | 1 M | 0.37 | 0.11 | 0.12 | 1.20 | 3.31 | 0.45 |
| BB-2 | 5.25 | 3 M | 0.58 | 0.12 | 0.21 | 1.45 | 4.00 | 1.14 |
| BB-2 | 5.25 | 6 M | 0.78 | 0.13 | 0.22 | 1.43 | 4.23 | 1.37 |
| BB-3 | 5.25 | Initial | 0.07 | 0.13 | 0.19 | 0.94 | 2.89 | — |
| BB-3 | 5.25 | 1 M | 0.36 | 0.13 | 0.12 | 1.07 | 3.19 | 0.30 |
| BB-3 | 5.25 | 3 M | 0.56 | 0.14 | 0.21 | 1.29 | 3.83 | 0.94 |
| BB-3 | 5.25 | 6 M | 0.75 | 0.14 | 0.21 | 1.36 | 4.06 | 1.17 |

TABLE 15
Stability Data of Daptomycin (350 mg & 500 mg) Containing L-Arginine HCl (210
mg & 300 mg) at Different pH Values under Long Term Conditions (25° C./60% RH)

| | | | | | | | Δ |
|---|---|---|---|---|---|---|---|
| | | | Lactone | Beta- | Anhydro | Total | (Total |
| Batch | Time Point | Impurity-1 | Hydrolysis | Isomer | daptomycin | Impurities | Impurities) |
| # | (Month, M) | (%) | (%) | (%) | (%) | (%) | (%) |
| AA-1 | Initial | 0.07 | 0.11 | 0.23 | 0.92 | 3.03 | — |
| AA-1 | 12 M | 0.11 | 0.44 | 0.25 | 1.24 | 3.80 | 0.77 |
| AA-1 | 18 M | 0.13 | 0.36 | 0.25 | 1.14 | 3.69 | 0.63 |
| AA-1 | 24 M | 0.41 | 0.13 | 0.25 | 1.26 | 3.80 | 0.70 |
| AA-2 | Initial | 0.08 | 0.12 | 0.20 | 0.96 | 2.91 | — |
| AA-2 | 12 M | 0.35 | 0.13 | 0.21 | 1.18 | 3.57 | 0.66 |
| AA-2 | 18 M | 0.41 | 0.14 | 0.21 | 1.21 | 3.69 | 0.78 |
| AA-2 | 24 M | 0.48 | 0.14 | 0.21 | 1.27 | 3.79 | 0.88 |
| AA-3 | Initial | 0.07 | 0.11 | 0.18 | 0.95 | 2.85 | — |
| AA-3 | 12 M | 0.35 | 0.12 | 0.21 | 1.16 | 3.47 | 0.62 |
| AA-3 | 18 M | 0.37 | 0.13 | 0.20 | 1.18 | 3.52 | 0.67 |
| AA-3 | 24 M | 0.44 | 0.12 | 0.20 | 1.27 | 3.69 | 0.84 |
| BB-1 | Initial | 0.09 | 0.10 | 0.20 | 0.97 | 2.79 | — |
| BB-1 | 12 M | 0.36 | 0.10 | 0.22 | 1.21 | 3.58 | 0.79 |
| BB-1 | 18 M | 0.45 | 0.10 | 0.21 | 1.19 | 3.69 | 0.9 |
| BB-1 | 24 M | 0.48 | 0.10 | 0.20 | 1.25 | 3.71 | 0.92 |
| BB-2 | Initial | 0.07 | 0.1 | 0.19 | 0.93 | 2.86 | — |
| BB-2 | 12 M | 0.35 | 0.11 | 0.20 | 1.21 | 3.68 | 0.82 |
| BB-2 | 18 M | 0.43 | 0.12 | 0.20 | 1.18 | 3.57 | 0.71 |
| BB-2 | 24 M | 0.46 | 0.12 | 0.20 | 1.23 | 3.65 | 0.79 |
| BB-3 | Initial | 0.07 | 0.13 | 0.19 | 0.94 | 2.89 | — |
| BB-3 | 12 M | 0.36 | 0.14 | 0.21 | 1.12 | 3.49 | 0.60 |
| BB-3 | 18 M | 0.43 | 0.14 | 0.21 | 1.12 | 3.53 | 0.64 |
| BB-3 | 24 M | 0.47 | 0.14 | 0.21 | 1.20 | 3.66 | 0.77 |

From Table 15, it can be seen that a daptomycin composition according to the present invention is stable for at least 24 months at 25° C./60% RH. A lyophilized composition containing daptomycin and L-arginine HCl in a molar ratio of about 1:4.6 provides no more than about 1% of increase in total impurities from the initial time point up to 24 months when stored under long-term storage conditions (25° C./60% RH).

### Example 8: Control of Impurities Using Different Concentration of pH Adjusting Agents

Different concentrations of pH adjusting agents, such as NaOH, may be used to control the formation of impurities during the manufacturing of daptomycin compositions. For example, 2N NaOH generates less impurities when compared to higher concentrations, for example, 4N and 5N NaOH solutions. Daptomycin compositions prepared using 2N NaOH generated less impurities when compared to compositions prepared using 4N or 5N NaOH. The compositions were prepared according to the process described in Example 1.

TABLE 16
Impurity Formation in Daptomycin Compositions (Daptomycin
350 mg and L-Arginine HCl 210 mg) using Different
NaOH Concentrations at the Initial Time Point.

| | NaOH | % Impurity | | | |
|---|---|---|---|---|---|
| | Normality | A (Lactone | | | |
| Batch | for pH | Hydrolysis) + | % | % Total | Target |
| Number | adjustment | Impurity 1 | Anhydro | Impurities | pH |
| CC | 2N | 0.13 | 0.82 | 2.59 | 5.25 |
| P | 4N | 0.29 | 0.95 | 3.03 | 5.25 |
| DD | 2N | 0.4 | 0.77 | 2.65 | 6.8 |
| EE | 5N | 0.7 | 1.3 | 3.7 | 6.8 |

### Example 9: Stability Comparison with CUBICIN® and CUBICIN RF®

As shown in Table 17, the daptomycin compositions described in one embodiment of the present invention exhibited less than about 4% of total impurities at the 24 month time point stored under controlled room temperature conditions (25° C. and 60% RH). In comparison, commercially available formulations CUBICIN and CUBICIN RF showed about 6% of total impurities when stored at room temperature and analyzed within one month of their stated expiration date.

TABLE 17
Stability Comparison

| | Related Substances → | | | | |
|---|---|---|---|---|---|
| Condition | Lactone | | Anhydro | Beta- | Total |
| ↓ | Hydrolysis | Impurity 1 | daptomycin | Isomer | Impurities |

CUBICIN ® (Daptomycin for injection) 500 mg/vial
B. no: Lot 934775

| | | | | | |
|---|---|---|---|---|---|
| Room | 0.24 | 0.18 | 1.20 | 1.13 | 5.75 |
| Temperature | | | | | |

CUBICIN ® (Daptomycin for injection) 500 mg/vial
B. no: Lot 934760

| | | | | | |
|---|---|---|---|---|---|
| Room | 0.20 | 0.26 | 1.22 | 1.02 | 5.84 |
| Temperature | | | | | |

CUBICIN ® RF (Daptomycin for injection) 500 mg/vial
B. no: Lot T009408

| | | | | | |
|---|---|---|---|---|---|
| Room | 0.43 | 0.57 | 0.95 | 0.85 | 5.86 |
| Temperature | | | | | |

Batch: BB-1 (Maia batch No. 247101; 350 mg/vial)

| | | | | | |
|---|---|---|---|---|---|
| 25° C./ | 0.10 | 0.48 | 1.25 | 0.20 | 3.71 |
| 60% RH | | | | | |
| 24 Months | | | | | |

Batch: AA-1 (Maia batch No. 245101; 500 mg/vial)

| | | | | | |
|---|---|---|---|---|---|
| 25° C./ | 0.13 | 0.41 | 1.26 | 0.25 | 3.80 |
| 60% RH | | | | | |
| 24 months | | | | | |

### Example 10: Stability of Reconstituted and/or Admixed Daptomycin Solutions at Room Temperature

Daptomycin for injection (Batch numbers: AA-1, AA-2, AA-3; Daptomycin: 500 mg, L-arginine HCl: 300 mg; pH: 5.25) that had been stored at room temperature for approximately 27 months after manufacture was reconstituted using sterile water for injection.

The reconstituted daptomycin composition according to one embodiment of the present invention exhibits less than about 5.5% of total impurities when reconstituted with WFI and stored for 18 hours at room temperature in the original vial. In contrast, the reconstituted solution of CUBICIN® and CUBICIN® RF exhibit about 6% o to 6.5% o of total impurities. Data for the reconstituted daptomycin composition and CUBICIN® and CUBICIN® RF is provided in Table 18(a) below.

TABLE 18(a)
a) Reconstitution Stability in Vials at Room Temperature (20-25° C.)

| | | In-Use | Lactone | | Beta- | | Total |
|---|---|---|---|---|---|---|---|
| | | time | hydrolysis | Impurity 1 | Isomer | Anhydro | Impurities |
| Composition | Preparation | point | (%) | (%) | (%) | (%) | (%) |
| CUBICIN ® | Reconstituted to | Initial | 0.24 | 0.18 | 1.13 | 1.20 | 5.83 |
| 500 mg/vial | 50 mg/L with | 12 Hrs | 0.27 | 0.18 | 0.93 | 2.06 | 6.48 |
| Lot 934775 | 0.9% Sodium | | | | | | |
| Exp May 2022 | Chloride Injection | | | | | | |
| CUBICIN ® RF | Reconstituted to | Initial | 0.43 | 0.56 | 0.86 | 0.97 | 5.80 |
| 500 mg/vial | 50 mg/mL with | 24 Hrs | 0.56 | 0.56 | 1.06 | 1.26 | 6.34 |
| Lot T009408 | Water for Injection | | | | | | |
| Exp September | | | | | | | |
| 2022 | | | | | | | |
| AA-1 | Reconstituted to | Initial | 0.13 | 0.36 | 0.26 | 1.21 | 3.77 |
| Daptomycin | 50 mg/L with | 18 hrs | 0.20 | 0.35 | 0.32 | 2.77 | 5.57 |
| 500 mg/vial | WFI | | | | | | |
| AA-2 | Reconstituted to | Initial | 0.14 | 0.40 | 0.21 | 1.19 | 3.45 |
| Daptomycin | 50 mg/L with | 18 hrs | 0.22 | 0.39 | 0.28 | 2.64 | 5.14 |
| 500 mg/vial | WFI | | | | | | |
| AA-3 | Reconstituted to | Initial | 0.13 | 0.40 | 0.21 | 1.22 | 3.59 |
| Daptomycin | 50 mg/L with | 18 hrs | 0.20 | 0.39 | 0.28 | 2.68 | 5.29 |
| 500 mg/vial | WFI | | | | | | |

A syringe compatibility study was conducted using samples from Batch Nos. AA-1, AA-2 and AA-3 (daptomycin 500 mg/vial) that were stored at room temperature for approximately 27 months after manufacture. Samples were reconstituted to 50 mg/mL using sterile water for injection USP, transferred into 10 mL polypropylene syringes, and stored for up to 18 hours at room temperature (20-25° C.).

A daptomycin solution according to one embodiment of the present invention stored in a polypropylene syringe exhibits an enhanced stability profile (less than about 5.5% of total impurities when stored for 18 hours at room temperature) when compared to CUBICIN® RF solution (about 6% to 6.5% of total impurities), as shown in Table 18(b).

A daptomycin for injection formulation according to one embodiment of the present invention reconstituted with sterile water for injection and stored in a polypropylene syringe is stable for up to 24 hours at room temperature.

TABLE 18(b)
b) Reconstituted Solution Syringe Stability at Room Temperature

| | | In-Use | Lactone | | Beta- | | Total |
|---|---|---|---|---|---|---|---|
| | | time | Hydrolysis | Impurity 1 | Isomer | Anhydro | impurities |
| Product | Preparation | point | (%) | (%) | (%) | (%) | (%) |
| AA-1 | Reconstituted to | Initial | 0.14 | 0.42 | 0.22 | 1.23 | 3.61 |
| | 50 mg/ml with WFI | 18 hrs (27) | 0.20 | 0.35 | 0.32 | 2.79 | 5.61 |
| | and stored in syringe | | | | | | |
| AA-2 | Reconstituted to | Initial | 0.12 | 0.35 | 0.25 | 1.20 | 3.64 |
| | 50 mg/ml with WFI | 18 hrs (27) | 0.22 | 0.39 | 0.28 | 2.64 | 5.15 |
| | and stored in syringe | | | | | | |
| AA-3 | Reconstituted to | Initial | 0.12 | 0.39 | 0.28 | 2.66 | 5.25 |
| | 50 mg/ml with WFI | 18 hrs (27) | 0.20 | 0.39 | 0.28 | 2.66 | 5.25 |
| | and stored in syringe | | | | | | |
| CUBICIN ® RF | Reconstituted to | Initial | 0.43 | 0.55 | 0.86 | 0.97 | 5.76 |
| 500 mg/vial | 50 mg/ml with WFI | 24 hrs | 0.57 | 0.56 | 1.07 | 1.28 | 6.38 |
| Lot T009408 | and stored in syringe | | | | | | |
| Exp: September | | | | | | | |
| 2022 | | | | | | | |

A compatibility study was conducted on daptomycin for injection when diluted in 0.9% sodium chloride injection, USP in intravenous bags. The study was conducted on samples (Batch Nos. AA-1, AA-2 and AA-3; daptomycin: 500 mg, L-arginine HCl: 300 mg; pH: 5.25) that were approximately 27 months old and were reconstituted to 50 mg/mL in the vial with either sterile water for injection USP or 0.9% sodium chloride injection USP then diluted in 0.9% sodium chloride injection USP to concentrations of 3.0 mg/mL and 8.4 mg/mL. The admixture was prepared in a bag.

The admixture concentrations were selected to evaluate a range of concentrations based on certain dosing for bacterial indications, adult weight of 70 kg and a range of pediatric weights using US CDC average weights for males.

The diluted solution is stable up to 18 hours at room temperature. The diluted daptomycin composition according to one embodiment of the present invention exhibits less total impurities (less than about 6%) when compared to CUBICIN® and CUBICIN RF®. It should be noted that the label for CUBICTN® states that the reconstitution followed by diluted solution can be stored for a maximum period of 12 hours.

Table 18(c) compares the in-use stability of CUBICIN®, CUBICIN RF®, and daptomycin for injection formulation Batch AA-1, AA-2 and AA-3.

TABLE 18(c)
c) Admixture Diluent Solution Stability at Room Temperature

| | | In-Use | Lactone | | Beta- | | |
|---|---|---|---|---|---|---|---|
| | | time | Hydrolysis | Impurity 1 | Isomer | Anhydro | Total |
| Product | Preparation | point | (%) | (%) | (%) | (%) | (%) |
| AA-1 | Reconstituted | Initial | 0.12 | 0.35 | 0.26 | 1.19 | 3.77 |
| | solution diluted to 3 | 18 hrs | 0.20 | 0.35 | 0.33 | 2.98 | 5.84 |
| | mg/mL in 0.9% | (27) | | | | | |
| | Sodium Chloride | | | | | | |
| | Injection | | | | | | |
| AA-1 | Reconstituted | Initial | 0.12 | 0.35 | 0.24 | 1.18 | 3.61 |
| | solution diluted to | 18 hrs | 0.21 | 0.34 | 0.32 | 2.96 | 5.62 |
| | 8.4 mg/mL in 0.9% | (27) | | | | | |
| | Sodium Chloride | | | | | | |
| | Injection | | | | | | |
| AA-2 | Reconstituted | Initial | 0.15 | 0.40 | 0.21 | 1.19 | 3.51 |
| | solution diluted to 3 | 18 hrs | 0.22 | 0.39 | 0.28 | 2.83 | 5.36 |
| | mg/mL in 0.9% | (27) | | | | | |
| | Sodium Chloride | | | | | | |
| | Injection | | | | | | |
| AA-2 | Reconstituted | Initial | 0.14 | 0.41 | 0.22 | 1.27 | 3.73 |
| | solution diluted to | 18 hrs | 0.23 | 0.40 | 0.30 | 2.85 | 5.59 |
| | 8.4 mg/mL in 0.9% | (27) | | | | | |
| | Sodium Chloride | | | | | | |
| | Injection | | | | | | |
| AA-3 | Reconstituted | Initial | 0.13 | 0.39 | 0.21 | 1.21 | 3.61 |
| | solution diluted to 3 | 18 hrs | 0.21 | 0.39 | 0.29 | 2.86 | 5.52 |
| | mg/mL in 0.9% | (27) | | | | | |
| | Sodium Chloride | | | | | | |
| | Injection | | | | | | |
| AA-3 | Reconstituted | Initial | 0.13 | 0.38 | 0.19 | 1.22 | 3.47 |
| | solution diluted to | 18 hrs | 0.21 | 0.37 | 0.27 | 2.81 | 5.27 |
| | 8.4 mg/mL in 0.9% | (27) | | | | | |
| | Sodium Chloride | | | | | | |
| | Injection | | | | | | |
| CUBICIN ® | Reconstituted | Initial | 0.25 | 0.18 | 0.94 | 1.23 | 5.61 |
| 500 mg/vial | solution diluted to 3 | 12 Hrs | 0.27 | 0.18 | 0.94 | 2.13 | 6.56 |
| | mg/mL in 0.9% | | | | | | |
| | Sodium Chloride | | | | | | |
| | Injection* | | | | | | |
| | Reconstituted | Initial | 0.25 | 0.19 | 0.94 | 1.19 | 5.59 |
| | solution diluted to | 12 Hrs | 0.26 | 0.18 | 0.93 | 1.91 | 6.31 |
| | 8.4 mg/mL in 0.9% | | | | | | |
| | Sodium Chloride | | | | | | |
| | Injection | | | | | | |
| CUBICIN ® | Reconstituted | Initial | 0.43 | 0.57 | 0.85 | 0.96 | 5.84 |
| RF | solution diluted to 3 | 19 hrs | 0.50 | 0.57 | 0.94 | 1.30 | 6.31 |
| | mg/mL in 0.9% | | | | | | |
| | Sodium Chloride | | | | | | |
| | Injection* | | | | | | |
| | Reconstituted | Initial | 0.43 | 0.57 | 0.85 | 0.9512 | 5.86 |
| | solution diluted to | 19 Hrs | 0.51 | 0.58 | 0.96 | 1.25 | 6.49 |
| | 8.4 mg/mL in 0.9% | | | | | | |
| | Sodium Chloride | | | | | | |
| | Injection† | | | | | | |

*Pediatric patient (23 kg, age 7-11) dosed at 7 mg/kg diluted in 50 mL;
†Pediatric patient (21 kg, age 2-6) dosed at 12 mg/kg diluted in 25 mL

### Example 11: Stability of Reconstituted and/or Admixed Daptomycin Solutions at 2-8° C.

Daptomycin for injection (Batch numbers: AA-1, AA-2, AA-3; Daptomycin: 500 mg, L-arginine HCl: 300 mg; pH: 5.25) that had been stored at room temperature for approximately 27 months after manufacture was reconstituted using sterile water for injection.

The stability of the reconstituted daptomycin for injection is provided in Table 19(a).

The reconstituted solution obtained from the daptomycin composition according to one embodiment of the present invention shows less than about 5% of total impurities when stored in the original vial at 2-8° C. for 120 hours. In contrast, the reconstituted solution of CUBICIN® and CUBICIN RF® shows about 6% of total impurities in the initial time point as shown in Table 18(c).

TABLE 19(a)
a) Reconstitution Solution Stability in Vials at 2-8° C.

| | | In-Use | Lactone | | Beta- | Anhydro | |
|---|---|---|---|---|---|---|---|
| | | time | hydrolysis | Impurity 1 | Isomer | daptomycin | Total |
| Composition | Preparation | point | (%) | (%) | (%) | (%) | (%) |
| AA-1 | Reconstituted to | Initial | 0.13 | 0.36 | 0.26 | 1.21 | 3.77 |
| Daptomycin | 50 mg/L with | 120 hrs | 0.16 | 0.36 | 0.28 | 2.01 | 4.67 |
| 500 mg/vial | WFI | | | | | | |
| AA-2 | Reconstituted to | Initial | 0.14 | 0.40 | 0.21 | 1.19 | 3.45 |
| Daptomycin | 50 mg/L with | 120 hrs | 0.17 | 0.40 | 0.24 | 1.96 | 4.29 |
| 500 mg/vial | WFI | | | | | | |
| AA-3 | Reconstituted to | Initial | 0.13 | 0.40 | 0.21 | 1.22 | 3.59 |
| Daptomycin | 50 mg/L with | 120 hrs | 0.17 | 0.39 | 0.24 | 1.98 | 4.47 |
| 500 mg/vial | WFI | | | | | | |

A syringe compatibility study was conducted using samples from Batch Nos. AA-1, AA-2, AA-3 (daptomycin 500 mg/vial) that were stored at room temperature for approximately 27 months after manufacture. Samples were reconstituted to 50 mg/mL using sterile water for injection USP, transferred into 10 mL polypropylene syringes, and stored for up to 10 days at refrigerated temperature (2-8° C.).

Daptomycin for injection formulation according to one embodiment of the present invention reconstituted with sterile water for injection and stored in a polypropylene syringe is stable for up to 10 days at 2-8° C.

A daptomycin solution according to one embodiment of the present invention stored in a polypropylene syringe exhibits an enhanced stability profile (less than about 5.5 of total impurities when stored at 10 days at 2-8° C.), as shown in Table 19(b) when compared to CUBICIN® RF solution (about 6 to 6.5% o of total impurities), as shown in Table 18(b).

TABLE 19(b)
b) Reconstituted Solution Syringe Stability at 2-8° C.

| | | In-Use | Lactone | | Beta- | | |
|---|---|---|---|---|---|---|---|
| | | time | Hydrolysis | Impurity 1 | Isomer | Anhydro | Total |
| Product | Preparation | point | (%) | (%) | (%) | (%) | (%) |
| AA-1 | Reconstituted to | Initial | 0.14 | 0.42 | 0.22 | 1.23 | 3.61 |
| | 50 mg/ml with WFI | 10 days | 0.21 | 0.35 | 0.31 | 2.65 | 5.52 |
| | and stored in syringe | | | | | | |
| AA-2 | Reconstituted to | Initial | 0.12 | 0.35 | 0.25 | 1.20 | 3.64 |
| | 50 mg/ml with WFI | 10 days | 0.22 | 0.38 | 0.27 | 2.47 | 5.12 |
| | and stored in syringe | | | | | | |
| AA-3 | Reconstituted to | Initial | 0.12 | 0.40 | 0.21 | 1.19 | 3.55 |
| | 50 mg/ml with WFI | 10 days | 0.24 | 0.39 | 0.30 | 2.95 | 5.77 |
| | and stored in syringe | | | | | | |

The diluted solution using 0.900 NaCl injection is stable up to 10 days (exhibits less than about 5.5% o of total impurities) at 2-8° C., as shown in Table 19(c). However, daptomycin solution obtained from CUBICIN®“and CUBICIN RF® using 0.9% o NaCl exhibits about 5.50% of total impurities at the initial time point, as shown in Table 18(c).

TABLE 19(c)
c) Admixture Diluent Solution Stability at 2-8° C.

| | | In-Use | Lactone | | Beta- | | |
|---|---|---|---|---|---|---|---|
| | | time | Hydrolysis | Impurity 1 | Isomer | Anhydro | Total |
| Product | Preparation | point | (%) | (%) | (%) | (%) | (%) |
| AA-1 | Reconstituted | Initial | 0.13 | 0.38 | 0.26 | 1.22 | 3.85 |
| | solution diluted to | 10 days | 0.19 | 0.36 | 0.32 | 2.75 | 5.54 |
| | 3 mg/mL in 0.9% | | | | | | |
| | Sodium Chloride | | | | | | |
| | Injection | | | | | | |
| AA-1 | Reconstituted | Initial | 0.13 | 0.36 | 0.24 | 1.22 | 3.67 |
| | solution diluted to | 10 days | 0.20 | 0.35 | 0.32 | 2.85 | 5.50 |
| | 8.4 mg/mL in 0.9% | | | | | | |
| | Sodium Chloride | | | | | | |
| | Injection | | | | | | |
| AA-2 | Reconstituted | Initial | 0.14 | 0.43 | 0.21 | 1.22 | 3.52 |
| | solution diluted to | 10 days | 0.21 | 0.41 | 0.28 | 2.65 | 5.12 |
| | 3 mg/mL in 0.9% | | | | | | |
| | Sodium Chloride | | | | | | |
| | Injection | | | | | | |
| AA-2 | Reconstituted | Initial | 0.15 | 0.43 | 0.22 | 1.27 | 3.74 |
| | solution diluted to | 10 days | 0.22 | 0.41 | 0.30 | 2.74 | 5.37 |
| | 8.4 mg/mL in 0.9% | | | | | | |
| | Sodium Chloride | | | | | | |
| | Injection | | | | | | |
| AA-3 | Reconstituted | Initial | 0.13 | 0.41 | 0.21 | 1.22 | 3.64 |
| | solution diluted to | 10 days | 0.19 | 0.40 | 0.27 | 2.58 | 3.64 |
| | 3 mg/mL in 0.9% | | | | | | |
| | Sodium Chloride | | | | | | |
| | Injection | | | | | | |
| AA-3 | Reconstituted | Initial | 0.13 | 0.39 | 0.19 | 1.21 | 3.45 |
| | solution diluted to | 10 days | 0.20 | 0.38 | 0.27 | 2.66 | 5.10 |
| | 8.4 mg/mL in 0.9% | | | | | | |
| | Sodium Chloride | | | | | | |
| | Injection | | | | | | |

It is to be understood that while the invention has been described in conjunction with the preferred specific embodiments thereof, that the foregoing description and the examples that follow are intended to illustrate and not limit the scope of the invention. It will be understood by those skilled in the art that various changes may be made, and equivalents may be substituted without departing from the scope of the invention, and further that other aspects, advantages and modifications will be apparent to those skilled in the art to which the invention pertains. In addition to the embodiments described herein, the present disclosure contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention.

1. A lyophilized pharmaceutical composition comprising daptomycin, L-arginine or a pharmaceutically acceptable salt thereof, and, optionally, one or more pH adjusting agents, wherein
(i) the molar ratio of daptomycin to L-arginine or a salt thereof is about 1:3.8 to about 1:5.6,
(ii) the pH of the composition is about 4.5 to about 7, and
(iii) the composition exhibits no more than about 4% (by HPLC) increase of total impurities from an initial time point to 6 months when stored at 40° C. and 75% relative humidity.

2. The pharmaceutical composition of claim 1, wherein the pharmaceutical composition is free or substantially free of (a) any amino acid other than L-arginine or a pharmaceutically acceptable salt thereof, (b) calcium chloride, or (c) any combination of any of the foregoing.

3. A lyophilized pharmaceutical composition consisting of daptomycin, L-arginine or a pharmaceutically acceptable salt thereof, and, optionally, one or more pH adjusting agents, wherein
(i) the molar ratio of daptomycin to L-arginine or salt thereof is about 1:3.8 to about 1:5.6, and
(ii) the pH of the composition is about 4.5 to about 7.

4. The pharmaceutical composition of claim 3, wherein the composition exhibits no more than about 1.5% (by HPLC) increase of N-decanoyl-L-tryptophyl-D-asparaginyl-L-aspartyl-2-aminobutenoyl-glycyl-L-ornithyl-L-aspartyl-D-alanyl-L-aspartyl-glycyl-D-seryl-threo-3-methyl-L-glutamyl-3-anthraniloyl-L-alanine (Impurity 1) and no more than about 3% (by HPLC) increase of total impurities from an initial time point to 3 months when stored at 40° C. and 75% relative humidity.

5. The pharmaceutical composition of claim 3, wherein the composition exhibits less than about 2.0% (by HPLC) increase of the sum of N-decanoyl-L-tryptophyl-D-asparaginyl-L-aspartyl-L-threonyne-glycyl-L-ornithyl-L-aspartyl-D-alanyl-L-aspartyl-glycyl-D-seryl-threo-3-methyl-L-glutamyl-3-anthraniloyl-L-alanine (Impurity A, lactone hydrolysis impurity) and N-decanoyl-L-tryptophyl-D-asparaginyl-L-aspartyl-2-aminobutenoyl-glycyl-L-ornithyl-L-aspartyl-D-alanyl-L-aspartyl-glycyl-D-seryl-threo-3-methyl-L-glutamyl-3-anthraniloyl-L-alanine (Impurity 1) upon storage from an initial point to 3 months at 40° C. and 75% relative humidity.

6. The pharmaceutical composition of claim 3, wherein the composition exhibits less than about 1.3% (by HPLC) increase of the sum of N-decanoyl-L-tryptophyl-D-asparaginyl-L-aspartyl-L-threonyne-glycyl-L-ornithyl-L-aspartyl-D-alanyl-L-aspartyl-glycyl-D-seryl-threo-3-methyl-L-glutamyl-3-anthraniloyl-L-alanine (Impurity A, lactone hydrolysis impurity) and N-decanoyl-L-tryptophyl-D-asparaginyl-L-aspartyl-2-aminobutenoyl-glycyl-L-ornithyl-L-aspartyl-D-alanyl-L-aspartyl-glycyl-D-seryl-threo-3-methyl-L-glutamyl-3-anthraniloyl-L-alanine (Impurity 1) upon storage from an initial point to 1 month when stored at 40° C. and 75% relative humidity.

7. The pharmaceutical composition of claim 3, wherein the composition exhibits no more than about 1.5% increase of the sum of N-decanoyl-L-tryptophyl-D-asparaginyl-L-aspartyl-L-threonyne-glycyl-L-ornithyl-L-aspartyl-D-alanyl-L-aspartyl-glycyl-D-seryl-threo-3-methyl-L-glutamyl-3-anthraniloyl-L-alanine (Impurity A, lactone hydrolysis impurity) and N-decanoyl-L-tryptophyl-D-asparaginyl-L-aspartyl-2-aminobutenoyl-glycyl-L-ornithyl-L-aspartyl-D-alanyl-L-aspartyl-glycyl-D-seryl-threo-3-methyl-L-glutamyl-3-anthraniloyl-L-alanine (Impurity 1) upon storage from an initial point to 12 months stored at 25° C. and 60% relative humidity.

8. The pharmaceutical composition of claim 1, wherein the composition exhibits no more than about 2% (by HPLC) increase in total impurities upon storage from an initial time point to 24 months at 25° C. and 60% relative humidity.

9. The pharmaceutical composition of claim 1, wherein the composition exhibits no more than about 1.9% (by HPLC) increase in total impurities upon storage from an initial time point to 1 month at 40° C. and 75% relative humidity.

10. The pharmaceutical composition of claim 1, wherein
(iii) the pH of the composition is about 5 to about 6, and
(iv) the composition exhibits no more than about 1% (by HPLC) increase of total impurities upon storage from an initial time point to 24 months when stored at 25° C. and 60% relative humidity.

11. The pharmaceutical composition according to claim 1, wherein the salt of L-arginine is a hydrochloride salt.

12. The pharmaceutical composition according to claim 1, wherein the one or more pH adjusting agents comprises 1N HCl, 2N NaOH, or a combination thereof.

13. pharmaceutical composition according to claim 1, wherein the molar ratio of daptomycin to L-arginine or a salt thereof is about 1:4 to about 1:5.6.

14. The pharmaceutical composition according to claim 1, wherein the molar ratio of daptomycin to L-arginine or a salt thereof is about 1:4.5 to about 1:4.7.

15. The pharmaceutical composition according to claim 1, wherein the composition is reconstituted using sterile water for injection, bacteriostatic water for injection or 0.9% sodium chloride.

16. The pharmaceutical composition according to claim 15, wherein the reconstituted solution is diluted using 0.9% sodium chloride or lactated Ringer's solution.

17. The pharmaceutical composition according to claim 15, wherein the diluted solution contains less than about 5.5% total impurities when stored for about 18 hours at room temperature.

18. A solid lyophilized pharmaceutical composition consisting of (i) about 350 mg daptomycin, (ii) about 210 mg L-arginine HCl, (iii) optionally 2N sodium hydroxide and/or 1N HCl, and (iv) optionally a buffering agent, wherein the pH of the composition is about 5.25.

19. The pharmaceutical composition of claim 18, wherein the composition exhibits no more than about 1% (by HPLC) increase of total impurities from an initial time point to 24 months when stored at 25° C. and 60% relative humidity.

20. A solid lyophilized pharmaceutical composition consisting of (i) about 500 mg daptomycin, (ii) about 300 mg L-arginine HCl, (iii) optionally 2N sodium hydroxide and/or 1N HCl, and (iv) optionally a buffering agent, wherein the pH of the composition is about 5.25.

21. The pharmaceutical composition of claim 20, wherein the composition exhibits no more than about 0.75% (by HPLC) increase of impurity 1 and no more than about 1.5% (by HPLC) increase of total impurities from an initial time point to 6 months when stored at 40° C. and 75% relative humidity.

22-26. (canceled)

27. A reconstituted pharmaceutical product comprising the pharmaceutical composition of claim 1 and a pharmaceutically acceptable diluent.

28. The reconstituted pharmaceutical product of claim 27, wherein the pharmaceutically acceptable diluent is selected from sterile water for injection, bacteriostatic water for injection and 0.9% NaCl solution.

29. The reconstituted pharmaceutical composition according to claim 27, wherein the pharmaceutical composition, after reconstitution, contains less than about 5.5% total impurities for at least 5 days at 2-8° C. when stored in a vial, or for at least 10 days at 2-8° C. when stored in a syringe.

30. The reconstituted pharmaceutical composition according to claim 27, wherein, after dilution in 0.9% sodium chloride, the pharmaceutical composition is stable for at least 10 days at 2-8° C., when stored in an intravenous bag.

31. A method of treating a bacterial infection comprising administering to a subject in need thereof the reconstituted pharmaceutical product according to claim 27.

32. The method according to claim 31, wherein the reconstituted pharmaceutical product is administered by intravenous or subcutaneous administration.
